(19) United States
(12) Patent Application Publication
(10) Pub. No.: US 2024/0091250 A1
(43) Pub. Date: Mar. 21, 2024

(54) AMERICAN GINSENG ROOT FRACTIONS, PROCESSES OF THEIR PREPARATION AND USES THEREOF

(71) Applicant: Jacqueline J. Shan, Edmonton (CA)

(72) Inventor: Jacqueline J. Shan, Edmonton (CA)

(21) Appl. No.: 18/267,362

(22) PCT Filed: Dec. 14, 2021
(86) PCT No.: PCT/CA2021/051807

§ 371 Date: Jun. 14, 2023

Related U.S. Application Data
(60) Provisional application No. 63/125,607, filed on Dec. 15, 2020.

(51) Int. Cl. A61K 31/715; A61K 36/258; A61P 31/16; A61P 31/22

(52) U.S. Cl. CPC A61K 31/715; A61K 36/258; A61P 31/16; A61P 31/22; A61K 2236/333; A61K 2236/51

(57) ABSTRACT

The present application relates to fractions from a root portion of an American ginseng (Panax quinquefolius) plant, and in particular, an intermediate polysaccharide fraction, a neutral polysaccharide fraction, an acidic polysaccharide rich fraction and an acidic polysaccharide fraction and to processes for their preparation, compositions comprising them, and to their use in therapy. For example, the fractions of the application are useful in the treatment of diseases, disorders or conditions treatable by activating pattern recognition receptors such as Toll-like receptors (TLR) or treatable by activating the innate and adaptive immune response, such as a viral infection.

### RELATED APPLICATIONS

The present application claims the benefit of priority of co-pending U.S. provisional patent application No. 63/125,607 filed on Dec. 15, 2020, the contents of which are incorporated herein by reference in their entirety.

### FIELD

The present application relates to fractions from a root portion of an American (Panax quinquefolius) ginseng plant. In particular, it relates to a neutral polysaccharide fraction, an acidic polysaccharide rich fraction and to an acidic polysaccharide fraction and to processes for their preparation, to compositions comprising them, and to their use in therapy. In particular, it relates to fractions from a root portion of an American (Panax quinquefolius) ginseng plant and to their use in the treatment of diseases, disorders or conditions treatable by activating the innate and adaptive immune response, such as a viral infection.

### BACKGROUND

The mammalian immune system has evolved multiple, layered and interactive defensive systems to protect against infections. These have been broadly divided into innate immunity and adaptive immunity. Innate immunity is the first line of defense against microbial pathogens and acts almost immediately to limit early proliferation and spread of infectious agents through activation of phagocytic and antigen-presenting cells (APCs), such as dendritic cells (DCs) and macrophages, and initiation of inflammatory responses through the release of a variety of cytokines, chemokines and anti-microbial factors, such as interferons (IFNs) and defenses. Innate immunity is evolutionarily ancient and for many years its study was largely ignored by immunologists as relatively non-specific. For the most part, though, people are protected against infection by our innate immune system. If, on the other hand, infectious organisms penetrate innate immune defenses, people's innate defenses facilitate and guide the generation of adaptive immune responses that are directed against highly specific determinants that are uniquely expressed by the invading pathogen. These responses are dependent on rearrangement of specific antigen-receptor genes in B-cells and T-cells and result in production of high-affinity antigen-specific antibodies (humoral immunity) and T-cells or cell-mediated immunity. In general, antibodies facilitate removal, destruction or neutralization of extracellular pathogens and their toxins, whereas T-cell-mediated immune responses help eliminate or control intracellular pathogens. In contrast to innate immune responses, adaptive immune responses have the hallmark of specific immune memory.

Until recent years, key unanswered questions have concerned how the host innate immune system detects infection and how it discriminates between self and pathogens or infectious non-self. The discovery and characterization of the Toll-like receptor (TLR) family has provided great insight into innate immune recognition and established a key role of the innate immune system in host defense against infection (Akira et al., 2006; Hargreaves and Medzhitov, 2005; Kawai and Akira, 2006; Philpott and Girardin, 2004; Seth et al., 2006). The innate immune system uses multiple families of germline encoded pattern recognition receptors (PRRs) to detect infection and trigger a variety of antimicrobial defense mechanisms (Janeway and Medzhitov, 1998). These PRRs are evolutionarily highly conserved among species from plants and fruit flies to mammals. The strategy of innate immune recognition is based on the detection of highly conserved and essential structures present in many types of microorganisms and absent from host cells (Janeway, 1992; Janeway and Medzhitov, 1999). Since the targets of innate immune recognition are conserved molecular patterns, they are called pathogen associated molecular patterns (PAMPs). PAMPS have three important features that make them ideal targets for innate immune sensing. First, PAMPs are produced only by microorganisms and not by host cells. This is the basis for discrimination of self and infectious non-self. Second, PAMPs are conserved between microorganisms of a given class. This allows a limited number of PRRs to detect the presence of a large class of invading pathogens. For example, a pattern in lipopolysaccharide (LPS) allows a single PRR to detect the presence of any Gram-negative bacteria. Third, PAMPs are essential for microbial survival and any mutation or loss of PAMPs are either lethal for the organism or greatly reduces their adaptive fitness. These new insights into innate immune recognition are revolutionizing the understanding of immune defense, pathogenesis, and treatment and prevention of infectious diseases.

Toll-like receptors (TLRs) are a type of PRR that recognize PAMPs. Signaling by TLRs is complex and has been reviewed elsewhere (Akira and Takeda, 2004; O'Neill, 2006). Briefly, all TLRs with the exception of TLR3 signal through the adaptor molecule myeloid differentiation factor 88 (MyD88), a cytoplasmic protein containing a toll-IL-1 receptor (TIR) domain and a death domain. Ultimately, NF-κ13 and MAPKs are activated downstream of TRAF6 leading to production of proinflammatory cytokines and chemokines, such as TNF-α, IL-6, IL-1β and IL-12. In addition to MyD88, TLR3 and TLR4 signal through TRIF, another TIR-containing adaptor that is required for production of type I interferons and type I interferon-dependent genes. TLRs are expressed on a variety of immune and non-immune cells. Murine macrophages express TLR1-9, reflecting their importance in the initiation of proinflammatory responses. Plasmacytoid dendritic cells (pDCs) that produce large amounts of type I interferons during viral infections express TLR7 and 9. All conventional dendritic cells (DCs) in the mouse express TLR1, 2, 4, 6, 8 and 9, while TLR3 is confined to the CD8+ and CD4− CD8− DC subset (Iwasaki and Medzhitov, 2004). In humans, TLR9 expression is restricted to pDCs and B-cells (Bauer et al., 2001; Krug et al., 2001). TLR3 is not expressed in pDCs of mice or humans. In addition to the focus on expression on immune cells there is great interest in understanding expression of TLRs on mucosal epithelial cells (ECs) that serve as the first line of defense against most infections.

TLRs induce a range of responses depending on the cell type they are activated in (Ashkar and Rosenthal, 2002; Iwasaki and Medzhitov, 2004). For example, treatment of DCs with CpG DNA that acts through TLR9 activates the DCs to mature, including upregulation of MHC class II and co-stimulatory molecules, as well as production of proinflammatory cytokines, chemokines and enhancement of antigen presentation. Similarly, treatment of B-cells with CpG induces their activation and proliferation, secretion of antibody as well as IL-6 and IL-10 and the B-cells become resistant to apoptosis. Activation of immune cells via CpG DNA induces a Th1-dominated response.

The mechanisms by which PRRs mediate host defense against pathogens are the focus of intense research. Due to their ability to enhance innate immune responses, novel strategies to use ligands, natural or synthetic agonists or antagonists of PRRs, so called ‘innate immunologicals’, can be used as stand alone or in conjunction with other anti-viral agents to provide protection or treatment against infection with intracellular bacteria, parasites and viruses. Further, activation of innate immune system through PRRs such as TLRs using their respective ligands or agonists represents a strategy to enhance immune responses against specific pathogens, making signaling via PRRs excellent vaccine adjuvants.

Korean red ginseng has been shown to have anti-viral effects through immune modulation (Nguyen and Nguyen, 2019).

### SUMMARY

The Applicants have developed processes for preparing polysaccharide extracts and fractions from a root portion of an American (Panax quinquefolius) ginseng plant. In particular, the Applicants have produced an intermediate polysaccharide fraction, from which a neutral polysaccharide fraction and an acidic polysaccharide rich fraction and acidic polysaccharide fraction have been produced.

Accordingly, the present application includes a process of preparing an intermediate polysaccharide fraction from a root portion of an American (Panax quinquefolius) ginseng plant, the process comprising:
extracting the root portion from a root portion of an American ginseng (Panax quinquefolius) plant with a solvent mixture comprising about 50% to about 95% (v/v) C₁₋₃ alkylOH and water at temperature of about 10° C. to about 90° C. to produce a solvent mixture fraction and a first residue, wherein the ratio of the solvent mixture to the root portion is about 20 to about 1 (v/w) to about 1 to about 1 (v/w),
separating the first residue from the solvent mixture fraction;
extracting the first residue with water at a temperature of about 10° C. to about 100° C. to produce an aqueous fraction comprising the intermediate polysaccharide fraction and a second residue, wherein the ratio of the water to the root portion is about 20 to about 1 (v/w) to about 1 to about 1 (v/w), and
separating the aqueous fraction comprising the intermediate polysaccharide fraction from the second residue; and
optionally, drying or concentrating the aqueous fraction to produce the intermediate polysaccharide fraction.

The present application also includes a process of preparing a neutral polysaccharide fraction, the process comprising:
providing an intermediate polysaccharide fraction produced by the process described above;
dissolving the intermediate polysaccharide fraction water to produce an intermediate polysaccharide fraction solution wherein the intermediate polysaccharide fraction solution is about 1% to about 10% (w/w),
adding C₁₋₃ alkylOH to the intermediate polysaccharide fraction solution to produce a neutral polysaccharide fraction and a first supernatant wherein the ratio of the C₁₋₃ alkylOH to the intermediate polysaccharide fraction solution is about 1 to about 1 (v/v) to about 0.5 to about 1 (v/v),
separating the neutral polysaccharide fraction from the first supernatant to produce the neutral polysaccharide fraction; and
optionally further purifying the neutral polysaccharide fraction by repeating steps the previous three steps except using the neutral polysaccharide fraction.

The present application includes a process of preparing an acidic polysaccharide rich fraction, the process comprising:
providing an intermediate polysaccharide fraction produced by the process described above;
dissolving the intermediate polysaccharide fraction in water to produce an intermediate polysaccharide fraction solution wherein the intermediate polysaccharide fraction (PBG-002) solution is about 1% to about 10% (w/w),
adding C₁₋₃ alkylOH to the intermediate polysaccharide fraction solution to produce a neutral polysaccharide fraction and a first supernatant wherein the ratio of the C₁₋₃ alkylOH to the intermediate polysaccharide fraction solution is about 1 to about 1 (v/v) to about 0.5 to about 1 (v/v), separating the first supernatant from the neutral polysaccharide fraction;
adding C₁₋₃ alkylOH to the first supernatant to produce an acidic polysaccharide rich fraction and a second supernatant wherein the ratio of the 3 alkylOH to first supernatant is about 0.5 to about 1 (v/v) to about 8 to about 1 (v/v), separating the acidic polysaccharide rich fraction from the second supernatant to produce the acidic polysaccharide rich fraction.

The present application further includes a process of preparing an acidic polysaccharide fraction, the process comprising:
providing an acidic polysaccharide rich fraction produced by the process described above;
dissolving the acidic polysaccharide rich fraction in water to produce an acidic polysaccharide rich fraction solution wherein the acidic polysaccharide rich fraction solution is about 1% to about 10% (w/w), adding C₁₋₃ alkylOH to the acidic polysaccharide rich fraction solution to produce a precipitate and a third supernatant comprising the acidic polysaccharide fraction wherein the ratio of C₁₋₃ alkylOH to the acidic polysaccharide rich fraction solution about 1 to about 1 (v/v) to about 0.5 to about 1 (v/v), separating the third supernatant comprising the acidic polysaccharide fraction from the precipitate; and optionally, drying or concentrating the third supernatant to produce the acidic polysaccharide fraction.

Alternatively, the acidic polysaccharide rich fraction is purified using column chromatography to produce the acidic polysaccharide fraction. Accordingly, the present application also includes a process of preparing an acidic polysaccharide fraction, the process comprising:
providing an acidic polysaccharide rich fraction produced by a process described above;
fractionating the acidic polysaccharide rich fraction from a chromatographic column comprising a diethylaminoethyl (DEAE) anion exchange resin to produce an acidic polysaccharide eluent fraction, wherein the fractionating comprises
(i) dissolving the acidic polysaccharide rich fraction in water to produce an acidic polysaccharide rich fraction solution;
(ii) loading the acidic polysaccharide rich fraction solution on the chromatographic column comprising the anion exchange resin; and
(iii) eluting the acidic polysaccharide rich eluent fraction from the anion exchange resin using an ammonium acetate buffer to produce the acidic polysaccharide eluent fraction, wherein the eluting comprises a first step wherein the buffer is about 10 mM to about 100 mM of ammonium acetate, and a second step wherein the buffer comprises about 0.5M to about 1.5M of ammonium acetate; and
optionally, drying or concentrating the acidic polysaccharide eluent fraction to produce the acidic polysaccharide fraction.

The present application also includes an intermediate polysaccharide fraction produced by a process described above.

In an embodiment, the present application includes an intermediate polysaccharide fraction having a carbohydrate content comprising about 0.5 to about 4 mole % rhamnose (Rha), about 13 to about 17 mole % galacturonic acid (GalA), about 70 to about 74 mole % glucose (Glc), about 3 to about 7 mole % galactose (Gal) and about 3 to about 7 mole % arabinose.

In an embodiment, the present application also includes a neutral polysaccharide fraction produced by a process described above.

The present application also includes a neutral polysaccharide fraction having a carbohydrate content comprising about 0.1 to about 3 mole % rhamnose (Rha), about 3 to about 7 mole % galacturonic acid (GalA), about 87 to about 92 mole % glucose (Glc), about 1 to about 4 mole % galactose (Gal) and about 1 to about 5 mole % arabinose (Ara).

In an embodiment, the present application also includes an acidic polysaccharide rich fraction produced by a process described above.

In an embodiment, the present application also includes an acidic polysaccharide fraction produced by a processes described above.

The present application includes an acidic polysaccharide fraction having a carbohydrate content comprising about 2 to about 6 mole % rhamnose (Rha), about 43 to about 47 mole % galacturonic acid (GalA), about 10 to about 14 mole % glucose (Glc), about 18 to about 22 mole % galactose (Gal) and about 16 to about 20 mole % arabinose (Ara)

The present application also includes a composition comprising one or more fractions of the application and a carrier.

The present application includes a method for activating Toll-like receptor (TLR) function in a cell, either in a biological sample or in a patient, comprising administering an effective amount of one or more fractions of the application to the cell.

The present application further includes a method for stimulating the production of one or more chemokines in a cell, either in a biological sample or in a patient, comprising administering an effective amount of one or more fractions of the application to the cell.

The present application also includes a method of treating a disease, disorder or condition by activation of TLR function comprising administering a therapeutically effective amount of one or more fractions of the application to a subject in need thereof.

The present application also includes a method for activating an innate and/or adaptive innate response in a cell, either in a biological sample or in a patient, comprising administering an effective amount of one or more fractions of the application to the cell.

The present application also includes a method of treating a viral infection comprising administering a therapeutically effective amount of one or more fractions of the application to a subject in need thereof.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole

### BRIEF DESCRIPTION OF DRAWINGS

The embodiments of the application will now be described in greater detail with reference to the attached drawings in which:

FIG. 1 is a flowchart showing the process of the preparation of intermediate polysaccharide fraction (PBG-002), and process of the purification of neutral polysaccharide fraction (PBG-005), acidic polysaccharide rich fraction (PBG-003), and acidic polysaccharide fraction (PBG-007) therefrom.

FIG. 2 shows the stimulation of IL-8 production by TLR2 expressing cells.

FIG. 3 shows the stimulation of IL-8 production by TLR4 expressing cells.

FIG. 4A shows the effect of fractions of the application on H1N1 viral titre in macrophage cell culture. *p<0.05, **p<0.001. FIG. 4B shows the effect of fractions of the application on H1N1 viral titre in macrophage cell culture. *p<0.05, **p<0.001.

FIG. 5 shows the effect of fractions of the application on HSV-1 viral titre in mouse dermal fibroblast cultures. *p<0.05, **p<0.001.

FIG. 6 shows the effect of fractions of the application on HSV-1 viral titre in human skin fibroblast line cultures. *p<0.05, **p<0.001.

FIG. 7 shows the effect of fractions of the application on HSV-2 viral titre in mouse dermal fibroblast cultures. *p<0.05, **p<0.001.

FIG. 8 shows the effect of fractions of the application on HSV-2 viral titre in human skin fibroblast line cultures. *p<0.05, **p<0.001.

FIG. 9A and FIG. 9B show the effect of fractions of the application on HSV-2 viral titre in human skin fibroblast line cultures. *p<0.05, **p<0.001.

FIG. 10 shows the effect of fractions of the application on viral titre of vaginal washes from guinea pigs following HSV-2 IVAG challenge.

FIG. 11 shows the effect of fractions of the application on vaginal lesion score during primary HSV-2 infection.

FIG. 12A shows the effect of fractions of the application on the total cumulative recurrent vaginal lesion score post primary infection with HSV-2. FIG. 12B shows the effect of fractions of the application on the individual cumulative recurrent vaginal lesion number post primary infection with HSV-2.

FIG. 13 shows the effect of fractions of the application on the percent survival of guinea pigs following HSV-3 IVAG challenge.

FIG. 14 shows the effect of fractions of the application on the daily body weights of guinea pigs following HSV-2 IVAG challenge.

### DETAILED DESCRIPTION

### I. Definitions

Unless otherwise indicated, the definitions and embodiments described in this, and other sections are intended to be applicable to all embodiments and aspects of the present application herein described for which they are suitable as would be understood by a person skilled in the art.

The term “fractions of the application” or “fractions of the present application” and the like as used herein refers to intermediate polysaccharide fraction (PBG-002), neutral polysaccharide fraction (PBG-005), acidic polysaccharide rich fraction and/or acidic polysaccharide fraction or enantiomers, salts and/or solvates thereof.

The term “composition of the application” or “composition of the present application” and the like as used herein refers to a composition comprising one or more fractions of the application.

The term “and/or” as used herein means that the listed items are present, or used, individually or in combination. In effect, this term means that “at least one of” or “one or more” of the listed items is used or present. The term “and/or” with respect to salts and/or solvates thereof means that the fractions of the application of the application exist as individual salts and hydrates, as well as a combination of, for example, a salt of a solvate of a compound of the application.

As used in the present application, the singular forms “a”, “an” and “the” include plural references unless the content clearly dictates otherwise. For example, an embodiment including “a negative response” should be understood to present certain aspects with one negative, or two or more additional negative responses.

In embodiments comprising an “additional” or “second” component or effect, such as an additional or second adverse effect, the second effect as used herein is different from the other effects or first effect. A “third” adverse effect is different from the other, first, and second effects, and further enumerated or “additional” adverse effects are similarly different.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

In embodiments comprising an “additional” or “second” component, the second component as used herein is chemically different from the other components or first component. A “third” component is different from the other, first, and second components, and further enumerated or “additional” components are similarly different.

The term “salt” means an acid addition salt or a basic addition salt. The term “salts” embraces salts commonly used to form addition salts of free acids or free bases and those compatible with the treatment of plants.

The term “solvate” as used herein means a compound, or a salt of a compound, wherein molecules of a suitable solvent are incorporated in the crystal lattice. A suitable solvent is physiologically tolerable at the dosage administered.

The expression “disease, disorder or condition associated with activating an innate and/or adaptive innate response” as used herein refers to any disease, disorder or condition that is directly or indirectly treatable by activation of the innate and/or adaptive innate response.

The term “subject” as used herein includes all members of the animal kingdom including mammals, including mammals, and suitably refers to humans. Thus the methods and uses of the present application are applicable to both human therapy and veterinary applications.

The term “pharmaceutical composition” as used herein refers to a composition of matter for pharmaceutical use.

The term “pharmaceutically acceptable salt” means an acid addition salt or a basic addition salt suitable for, or compatible with, the treatment of subjects.

The term “pharmaceutically acceptable salts” embraces salts commonly used to form addition salts of free acids or free bases.

As used herein, the term “effective amount” or “therapeutically effective amount” means an amount effective, at dosages and for periods of time necessary to achieve a desired result.

The terms “to treat”, “treating” and “treatment” as used herein and as is well understood in the art, means an approach for obtaining beneficial or desired results, including clinical results. Examples of beneficial or desired clinical results include, but are not limited to, diminishment of extent of viral or microbial infection, stabilization (i.e. not worsening) of the state of the viral or microbial infection, preventing spread of the viral or microbial infection, delay or slowing of infection progression, amelioration or palliation of the viral or microbial infectious state, diminishment of the reoccurrence of viral infection, diminishment, stabilization, alleviation or amelioration of one or more diseases, disorders or conditions arising from the viral or microbial infection, diminishment of the reoccurrence of one or more diseases, disorders or conditions arising from the viral or microbial infection, and remission of the viral or microbial infection and/or one or more symptoms or conditions arising from the viral or microbial infection, whether partial or total, whether detectable or undetectable. “To treat”, “treating” and “treatment” can also mean prolonging survival as compared to expected survival if not receiving treatment. “To treat”, “treating” and “treatment” as used herein also include prophylactic treatment. For example, a subject with an early viral or microbial infection is treated to prevent progression, or alternatively a subject in remission is treated to prevent recurrence.

“Palliating” an infection, disease, disorder and/or condition means that the extent and/or undesirable clinical manifestations of an infection, disease, disorder and/or condition are lessened and/or time course of the progression is slowed or lengthened, as compared to not treating the infection, disease, disorder and/or condition.

The term “viral infection” as used herein refers to an invasion of cells or bodily tissues by one or more foreign, undesirable viruses.

The term “fraction” refers to a concentrated preparation of plant material that has been produced by extraction or precipitation of the plant material with a suitable solvent or mixture of solvents.

The term “intermediate fraction” refers to a concentrated preparation of plant material that has been produced by extraction or precipitation of the plant material with a suitable solvent or mixture of solvents from which additional “fractions” can be produced.

The term “nutraceutical” or “functional food” as used herein refers to a natural, bioactive chemical compound that provides a physiological benefit, for example, disease prevention or treatment and/or health promotion and which may be used to supplement the diet.

The term “PBG-002” as used herein refers to the intermediate polysaccharide fraction of the application.

The term “PBG-005” as used herein refers to the neutral polysaccharide fraction of the application.

The term “PBG-003” as used herein refers to the acidic polysaccharide rich fraction of the application.

The terms “PBG-007” or “PBG-007-1” or “PBG-007-2” as used herein refer to the acidic polysaccharide fraction of the application.

When used, for example, with respect to the methods of treatment, uses, compositions and kits of the application, a subject, for example a subject “in need thereof” is a subject who has been diagnosed with, is suspected of having, may come in to contact with, and/or was previously treated fora viral or microbial infection or a disease, disorder or condition arising from a viral or microbial infection.

### II. Processes of Preparing Fractions of the Application

The Applicants have developed processes of preparing and obtaining polysaccharide extracts or fractions from a root portion of an American (Panax quinquefolius) ginseng plant. In particular, the Applicants have obtained an intermediate polysaccharide fraction (PBG-002), from which a neutral polysaccharide fraction (PBG-005), and an acidic polysaccharide rich fraction (PBG-003), acidic polysaccharide fraction (PBG-007) have been obtained. The intermediate polysaccharide fraction, neutral polysaccharide fraction, acidic polysaccharide rich fraction and acidic polysaccharide fraction have been shown to activate Toll-like receptors (TLR) and stimulate the production of one or more chemokines. Therefore, the intermediate polysaccharide fraction, neutral polysaccharide fraction, acidic polysaccharide rich fraction and acidic polysaccharide fraction have been shown to be TLR agonists and immunological agents, for example, immunostimulators, which can activate the innate and adaptive immune system, and in particular, the innate immune system. The intermediate polysaccharide fraction, neutral polysaccharide fraction, acidic polysaccharide rich fraction and acidic polysaccharide fraction have also been shown have a significant beneficial effect on virus titre and thus have been shown to confer protection against viral infection, in both in vitro and in vivo models. Therefore, the intermediate polysaccharide fraction, neutral polysaccharide fraction, acidic polysaccharide rich fraction and acidic polysaccharide fraction have been shown to have antiviral activity.

Accordingly, the present application includes a process of preparing an intermediate polysaccharide fraction (PBG-002) from a root portion of an American ginseng (Panax quinquefolius) plant, the process comprising:
extracting the root portion from a root portion of an American ginseng (Panax quinquefolius) plant with a solvent mixture comprising about 50% to about 95% (v/v) C₁₋₃ alkylOH and water at temperature of about 10° C. to about 90° C. to produce a solvent mixture fraction and a first residue, wherein the ratio of the solvent mixture to the root portion is about 20 to about 1 (v/w) to about 1 to about 1 (v/w),
separating the first residue from the solvent mixture fraction;
extracting the first residue with water at a temperature of about 10° C. to about 100° C. to produce an aqueous fraction comprising the intermediate polysaccharide fraction and a second residue, wherein the ratio of the water to the root portion is about 20 to about 1 (v/w) to about 1 to about 1 (v/w), and
separating the aqueous fraction comprising the intermediate polysaccharide fraction from the second residue; and
optionally, drying or concentrating the aqueous fraction to produce the intermediate polysaccharide fraction.

It would be appreciated by a person skilled in the art that the root portion is any portion from the root of the plant. In an embodiment, the root portion is a fresh root portion. In an embodiment, the root portion is a dried root portion. In an embodiment, the dried root portion is white ginseng. In an embodiment, the dried root portion is red ginseng.

In an embodiment, the root portion is comminuted before of extracting the root portion to produce a comminuted root portion. Therefore, the root portion is a comminuted root portion. In an embodiment, the comminuted root portion is micronized. Therefore, the comminuted root portion is a micronized root portion. In an embodiment, micronized root portion is micronized by milling, bashing or grinding. In an embodiment, the root portion is micronized by milling. Therefore, in an embodiment, the root portion is a milled root portion. In an embodiment, the root portion is a pulverized root portion.

In an embodiment, the root portion is available worldwide from a number of commercial sources. Commercial sources include, for example, department stores, road shops, large retailers, supermarkets, duty-free shops, drugstores, traditional markets, and internet retail sources.

In an embodiment, the root portion has a particle size range of about 100 μm to about 1500 μm, about 100 μm to about 1000 μm, about 150 μm to about 900 μm, about 150 μm to about 800 μm, about 200 μm to about 600, or about 200 μm to about 450 μm.

In an embodiment, the solvent mixture in step of extracting the root portion comprises about 55% C₁₋₃ alkylOH (v/v) to about 95% C₁₋₃ alkylOH (v/v), about 60% C₁₋₃ alkylOH (v/v) to about 95% C₁₋₃ alkylOH (v/v), about 70% C₁₋₃ alkylOH (v/v) to about 95% C₁₋₃ alkylOH(v/v), about 75% C₁₋₃ alkylOH (v/v) to about 90% 3 alkylOH (v/v), about 80% C₁₋₃ alkylOH (v/v) to about 90% C₁₋₃ alkylOH (v/v), about 80% C₁₋₃ alkylOH (v/v) to about 85% C₁₋₃ alkylOH (v/v). In an embodiment, the solvent mixture comprises about 60% C₁₋₃ alkylOH (v/v) to about 95% C₁₋₃ alkylOH (v/v), about 75% C₁₋₃ alkylOH (v/v) to about 90% C₁₋₃ alkylOH (v/v), or about 80% 3 alkylOH (v/v) to about 90% C₁₋₃ alkylOH (v/v). In an embodiment, the solvent mixture comprises about 80% C₁₋₃ alkylOH (v/v) to about 90% C₁₋₃ alkylOH (v/v). In an embodiment, the solvent mixture comprises about 50% C₁₋₃ alkylOH (v/v), about 60% C₁₋₃ alkylOH(v/v), about 70% C₁₋₃ alkylOH (v/v), about 75% C₁₋₃ alkylOH (v/v), about 80% C₁₋₃ alkylOH (v/v), about 85% C₁₋₃ alkylOH (v/v), about 90% C₁₋₃ alkylOH (v/v) or about 95% C₁₋₃ alkylOH (v/v). In an embodiment, the solvent mixture comprises about 75% C₁₋₃ alkylOH (v/v), about 80% C₁₋₃ alkylOH (v/v), about 85% C₁₋₃ alkylOH (v/v) about 90 C₁₋₃ alkylOH (v/v) or about 95% C₁₋₃ alkylOH (v/v). In an embodiment, the solvent mixture comprises about 85% C₁₋₃ alkylOH (v/v). In an embodiment, the solvent mixture comprises greater than about 75% C₁₋₃ alkylOH (v/v), greater than about 80% C₁₋₃ alkylOH (v/v), greater than about 85% C₁₋₃ alkylOH (v/v) greater than about 90 C₁₋₃ alkylOH (v/v) or greater than about 95% C₁₋₃ alkylOH (v/v). In an embodiment, the solvent mixture comprises a greater than about 85% C₁₋₃ alkylOH (v/v).

In an embodiment, the C₁₋₃ alkylOH in the step of extracting the root portion is selected from methanol, ethanol, propanol and isopropanol. In an embodiment, the C₁₋₃ alkylOH is selected form methanol and ethanol. In an embodiment, the C₁₋₃ alkylOH is ethanol.

In an embodiment, the ratio of the solvent mixture to the root portion in the step of extracting the root portion is about 18 to about 1 (v/w), about 15 to about 1 (v/w), about 10 to about 1 (v/w), about 5 to about 1 (v/w), or about 1 to about 1 (v/w). In an embodiment, the ratio of the solvent mixture to the root portion in the step of extracting the root portion is about 15 to about 1 (v/w) to 5 to about 1 (v/w). In an embodiment, the ratio of the solvent mixture to the root portion in the step of extracting the root portion is about 10 to about 1 (v/w). In an embodiment, the (v/w) is L/Kg.

In an embodiment, the step of extracting the root portion with a solvent mixture is performed at a temperature of about 20° C. to about 90° C., about 30° C. to about 90° C., about 40° C. to about 90° C., about 50° C. to about 90° C., about 50° C. to about 60° C., about 50° C. to about 70° C., about 50° C. to about 80° C., about 60° C. to about 70° C. about 60° C. to about 80° C., about 70° C. to about 80° C., about 50° C. to about 90° C., about 60° C. to about 90° C., about 70° C. to about 90° C., about 80° C. to about 90° C. In an embodiment, the step of extracting the root portion from a root portion of an American ginseng (Panax quinquefolius) plant with a solvent mixture is performed at a temperature of about 20° C. to about 90° C., about 30° C. to about 90° C., about 40° C. to about 90° C., about 50° C. to about 90° C., about 60° C. to about 90° C., about 70° C. to about 90° C., or about 80° C. to about 90° C. In an embodiment the step of extracting the root portion with a solvent mixture is performed at the boiling point of the solvent mixture.

It would be appreciated by a person skilled in the art that the step of extracting the root portion with a solvent mixture should be for a length of time to allow for sufficient extraction of undesired materials from the first residue. In an embodiment, the step of extracting the root portion with a solvent mixture is performed over more than about 1 hour, more than about 2 hours, more than about 3 hours, more than about 4 hours, or more than about 5 hours. In an embodiment, the step of extracting the root portion with a solvent mixture is performed over more than about 1 hour, more than about 2 hours, or more than about 3 hours. In an embodiment, the step of extracting the root portion with a solvent mixture is performed over about 1 hour to about 5 hours, about 2 hours to about 3 hours, about 2 hours to about 4 hours, about 1 hour to about 4 hours or about 1 hour to about 3 hours. In an embodiment, the step of extracting the root portion with a solvent mixture is performed over about 1 hour to about 3 hours. In an embodiment, the step of extracting the root portion with a solvent mixture is performed over about 3 hours.

In an embodiment, the ratio of the water to the root portion in the step of extracting the first residue is about 18 (v/w) to about 1 (v/w), about 15 (v/w) to about 1 (v/w), about 12 (v/w) to about 1 (v/w), about 10 (v/w) to about 1 (v/w), about 5 (v/w) to about 1 (v/w), or about 1 (v/w) to about 1 (v/w). In an embodiment, the ratio the water to the root portion in the step of extracting the first residue is about 15 (v/w) to about 1 (v/w) to about 5 (v/w) to about 1 (v/w). In an embodiment, the ratio is about 12 (v/w) to about 1 (v/w).

In an embodiment, the step of extracting the first residue with water is performed at a temperature of about 20° C. to about 100° C., about 30° C. to about 100° C., about 40° C. to about 100° C., about 50° C. to about 100° C., about 60° C. to about 100° C., 20° C. to about 90° C., about 30° C. to about 90° C., about 40° C. to about 90° C., about 50° C. to about 90° C., about 50° C. to about 60° C., about 50° C. to about 70° C., about 50° C. to about 80° C., about 60° C. to about 70° C. about 60° C. to about 80° C., about 70° C. to about 80° C., about 50° C. to about 90° C., about 60° C. to about 90° C., about 70° C. to about 90° C., about 80° C. to about 90° C., about 70° C. to about 100° C., about 80° C. to about 100° C. or about 90° C. to about 100° C. In an embodiment, the step of extracting the first residue with water is performed at about 80° C. to about 100° C. In an embodiment, the step of extracting the first residue with water in is performed at about 100° C.

It would be appreciated by a person skilled in the art that the step of extracting the first residue with water should be for a length of time to allow for sufficient extraction of the intermediate polysaccharide fraction. In an embodiment, the step of extracting the first residue with water is performed over more than about 1 hour, more than about 2 hours, more than about 3 hours, more than about 4 hours, or more than about 5 hours. In an embodiment, the step of extracting the first residue with water is performed over more than about 1 hour, more than about 2 hours, or more than about 3 hours. In an embodiment, the step of extracting the first residue with water is performed over about 1 hour to about 5 hours, about 2 hours to about 3 hours, about 2 hours to about 4 hours, about 1 hour to about 4 hours or about 1 hour to about 3 hours. In an embodiment, the step of extracting the first residue with water is performed over about 1 hour to about 3 hours. In an embodiment, the step of extracting the first residue with water is performed over more than about 1 hour. In an embodiment, the step of extracting the first residue with water is performed over about 3 hours.

It would be appreciated by a person skilled in the art that the first residue can be separated from the solvent mixture fraction and/or the aqueous fraction can be separated from the second residue using any method of separating that is known in the art. In an embodiment, the separating the aqueous fraction is by filtering, decanting or centrifugation. In an embodiment, the separating is by centrifugation.

In an embodiment, the centrifugation is performed at about 800 rpm to about 1500 rpm, about 1000 rpm to about 1500 rpm, or about 1100 rpm to about 1300 rpm. In an embodiment, the centrifugation is performed at about 1200 rpm. In an embodiment, the centrifugation is performed over about 5 minutes to about 20 minutes, for about 5 minutes to about 15 minutes, or for about 8 minutes to about 12 minutes. In an embodiment, the centrifugation is performed over about 10 minutes.

In an embodiment, the step of drying or concentrating is by any method of solvent removal known in the art. In an embodiment, the step of drying or concentrating is by evaporation. In an embodiment, the evaporation is by distillation, open air evaporation, evaporation under vacuum, or rotoevapouration. In an embodiment, the evaporation is by rotoevapouration.

In an embodiment, the step of drying comprises concentrating the aqueous fraction comprising the intermediate polysaccharide followed by freeze drying or spray drying to produce the intermediate polysaccharide. In an embodiment, the drying comprises concentrating the aqueous fraction comprising the intermediate polysaccharide followed by freeze drying to produce the intermediate polysaccharide.

It would be appreciated by a person skilled in the art that “optionally’ in step of optionally, drying or concentrating the aqueous fraction to produce the intermediate polysaccharide fraction means that the aqueous fraction comprising the intermediate polysaccharide fraction can be used as produced after the step of separating the aqueous fraction comprising the intermediate polysaccharide fraction from the second residue, or the aqueous fraction can be further dried or concentrated as described above.

In an embodiment, the intermediate polysaccharide fraction comprises a neutral polysaccharide fraction and an acidic polysaccharide rich fraction from which an acidic polysaccharide fraction is further obtained. In an embodiment, the polysaccharide extract further comprises saponins and/or oligosaccharides.

In an embodiment, the intermediate polysaccharide fraction produced after drying is a solid, an oil, a wax or a gum. In an embodiment, the intermediate polysaccharide fraction is a solid. In an embodiment, the intermediate polysaccharide fraction is a solid, and the solid is a powder.

In an embodiment, the present application also includes a process of preparing an intermediate polysaccharide fraction (PGB-002) from a root portion of an American ginseng (Panax quinquefolius) plant, the process comprising:
extracting the root portion from a root portion of an American ginseng (Panax quinquefolius) plant with a solvent mixture comprising about 50% (v/v) to about 95% (v/v) ethanol and water at a temperature of about 10° C. to about 90° C. to produce a solvent mixture fraction and a first residue, wherein the ratio of the solvent mixture to the root portion is about 20 (v/w) to about 1 (v/w) to about 1 (v/w) to about 1 (v/w),
separating the first residue from the solvent mixture fraction;
extracting the first residue with water at a temperature of about 10° C. to about 100° C. to produce an aqueous fraction comprising the intermediate polysaccharide fraction and a second residue, wherein the ratio of the water to the root portion is about 20 (v/w) to about 1 (v/w) to about 1 (v/w) to about 1 (v/w),
separating the aqueous fraction comprising the intermediate polysaccharide fraction from the second residue; and
optionally, drying or concentrating the aqueous fraction to produce the intermediate polysaccharide fraction.

The Applicants have further obtained a neutral polysaccharide fraction (PBG-005), and an acidic polysaccharide rich fraction (PBG-003) from the intermediate polysaccharide.

Accordingly, the present application also includes a process of preparing a neutral polysaccharide fraction (PBG-005), the process comprising:
providing an intermediate polysaccharide fraction produced by the process described above;
dissolving the intermediate polysaccharide fraction in water to produce an intermediate polysaccharide fraction solution wherein the intermediate polysaccharide fraction solution is about 1% to about 10% (w/w),
adding C₁₋₃ alkylOH to the intermediate polysaccharide fraction solution to produce a neutral polysaccharide fraction and a first supernatant, wherein the ratio of the C₁₋₃ alkylOH to the intermediate polysaccharide fraction solution is about 1 to about 1 (v/v) to about 0.5 to about 1 (v/v),
separating the neutral polysaccharide fraction from the first supernatant to produce the neutral polysaccharide fraction; and
optionally further purifying the neutral polysaccharide fraction by repeating the previous three steps except using the neutral polysaccharide fraction.

In an embodiment, the intermediate polysaccharide fraction solution is about 2 (w/w) % to about 10% (w/w), about 3% to about 10% (w/w), about 4% to about 10% (w/w), about 5% to about 10% (w/w), about 4% to about 9% (w/w), about 4% to about 8% (w/w), about 4% to about 7% (w/w), about 4% to about 6% (w/w), about 2% (w/w), about 3% (w/w), about 4% (w/w), about 5% (w/w), or about 6% (w/w). In an embodiment, the intermediate polysaccharide fraction solution is about 5% (w/w).

In an embodiment, the C₁₋₃ alkylOH in the step of adding C₁₋₃ alkylOH to the intermediate polysaccharide fraction solution is selected from methanol, ethanol, propanol and isopropanol. In an embodiment, the C₁₋₃ alkylOH is selected from methanol and ethanol. In an embodiment, the C₁₋₃ alkylOH is ethanol.

In an embodiment, the ratio of C₁₋₃ alkylOH to the intermediate polysaccharide fraction solution is about 1 (v/v) to about 1 (v/v) to about 0.5 (v/v) to about 1 (v/v). In an embodiment, the ratio of C₁₋₃ alkylOH to the intermediate polysaccharide fraction solution is about 1 (v/v) to about 1 (v/v) to about 0.5 (v/v) to about 1 (v/v) or about 0.7 (v/v). to about 1 (v/v) to about 0.5 to about 1 (v/v). In an embodiment, the ratio of C₁₋₃ alkylOH to the intermediate polysaccharide fraction solution is about 0.7 (v/v). to about 1 (v/v) to about 0.5 to about 1 (v/v). In an embodiment, the ratio of C₁₋₃ alkylOH to the intermediate polysaccharide fraction solution is about 0.5 (v/v) to about 1 (v/v).

In an embodiment, the step of adding C₁₋₃ alkylOH to the intermediate polysaccharide fraction solution to produce a neutral polysaccharide fraction and a first supernatant is performed over more than about 1 hour, more than about 2 hours, more than about 3 hours, more than about 4 hours, more than about 5 hours or more than about 6 hours. In an embodiment, the step of adding C₁₋₃ alkylOH to the intermediate polysaccharide fraction solution to produce a neutral polysaccharide fraction and a first supernatant is performed over more than about 1 hour, more than about 2 hours, or more than about 3 hours. In an embodiment, the step of adding 3 alkylOH to the intermediate polysaccharide fraction solution to produce a neutral polysaccharide fraction and a first supernatant is performed over about 1 hour to about 5 hours, about 2 hours to about 3 hours, about 2 hours to about 4 hours, about 1 hour to about 4 hours or about 1 hour to about 3 hours. In an embodiment, the step of adding C₁₋₃ alkylOH to the intermediate polysaccharide fraction solution to produce a neutral polysaccharide fraction and a first supernatant is performed over about 1 hour to about 3 hours. In an embodiment, the step of adding C₁₋₃ alkylOH to the intermediate polysaccharide fraction solution to produce a neutral polysaccharide fraction and a first supernatant is performed over about 3 hours after the adding the C₁₋₃ alkylOH to the intermediate polysaccharide fraction solution. In an embodiment, the step of adding C₁₋₃ alkylOH to the intermediate polysaccharide fraction solution to produce a neutral polysaccharide fraction and a first supernatant is performed over more than about 1 hour.

In an embodiment, the neutral polysaccharide fraction is optionally further purified by repeating the previous three steps in the process as described above except using the neutral polysaccharide fraction to produce a purified neutral polysaccharide fraction. In an embodiment, the neutral polysaccharide fraction is optionally further purified by repeating the steps of “dissolving the intermediate polysaccharide fraction in water to the step of separating the neutral polysaccharide fraction from the first supernatant to produce the neutral polysaccharide fraction, except that the neutral polysaccharide fraction is used in place of the intermediate polysaccharide fraction to produce a purified neutral polysaccharide fraction. In an embodiment, the neutral polysaccharide fraction is further purified by repeating the previous three steps one time. In an embodiment, the neutral polysaccharide fraction is further purified by repeating the previous three steps at least one time.

It would be appreciated by a person skilled in the art that the neutral polysaccharide fraction can be separated from the first supernatant using any method known in the art. In an embodiment, the separating is by filtering, decanting or centrifugation. In an embodiment, the separating is by centrifugation.

In an embodiment, the neutral polysaccharide fraction is a solid, an oil, a wax or a gum. In an embodiment, the neutral polysaccharide fraction is a solid. In an embodiment, the neutral polysaccharide fraction is a solid, and the solid is a powder.

In an embodiment, the process provides the neutral polysaccharide fraction in a purity of between about 80% to about 100%, about 85% to about 100%, about 90% to about 100%, about 95% to about 100%, about 95% to about 98% or about 95% (w/w) or more after purifying the produced neutral polysaccharide fraction by repeating the previous three steps as described above one time.

In an embodiment, the process provides the neutral polysaccharide fraction in an overall yield of about 10% to about 15% after the produced neutral polysaccharide fraction is purified by repeating the previous three steps as described above one time when the intermediate polysaccharide fraction is taken as the initial starting material.

The present application includes a process of preparing a neutral polysaccharide fraction (PBG-005), the process comprising:
providing an intermediate polysaccharide fraction produced by the process described above;
dissolving the intermediate polysaccharide fraction in water to produce an intermediate polysaccharide fraction solution, wherein the intermediate polysaccharide fraction solution is about 1% (w/w) to about 10% (w/w), adding ethanol to the intermediate polysaccharide fraction solution to produce a neutral polysaccharide fraction and a first supernatant, wherein the ratio of the ethanol to the intermediate polysaccharide fraction solution is about 1 (v/v) to about 1 (v/v) to about 0.5 (v/v) to about 1 (v/v);
separating the neutral polysaccharide fraction from the first supernatant to produce the neutral polysaccharide fraction; and
optionally purifying the neutral polysaccharide fraction by repeating the previous three steps.

The present application includes a process of preparing a neutral polysaccharide fraction (PGB-005) from a root portion of an American ginseng (Panax quinquefolius) plant, the process comprising:
extracting the root portion from a root portion of an American ginseng (Panax quinquefolius) plant with a solvent mixture comprising about 50% (v/v) to about 95% (v/v) C₁₋₃ alkylOH and water at a temperature of about 10° C. to about 90° C. to produce a solvent mixture fraction and a first residue, wherein the ratio of the solvent mixture to the root portion is about 20 (v/w) to about 1 (v/w) to about 1 (v/w) to about 1 (v/w),
separating the first residue from the solvent mixture fraction;
extracting the first residue with water at a temperature of about 10° C. to about 100° C. to produce an aqueous fraction comprising the intermediate polysaccharide fraction and a second residue, wherein the ratio of the water to the root portion is about 20 (v/w) to about 1 (v/w) to about 1(v/w) to about 1 (v/w),
separating the aqueous fraction comprising the intermediate polysaccharide fraction from the second residue;
drying or concentrating the aqueous fraction to produce the intermediate polysaccharide fraction;
dissolving the intermediate polysaccharide fraction in water to produce an intermediate polysaccharide fraction solution, wherein the intermediate polysaccharide fraction solution is about 1% (w/w) to about 10% (w/w),
adding C₁₋₃ alkylOH to the intermediate polysaccharide fraction solution to produce a neutral polysaccharide fraction and a first supernatant wherein the ratio of the C₁₋₃ alkylOH to the intermediate polysaccharide fraction solution is about 1 (v/v) to about 1 (v/v) to about 0.5 (v/v) to about 1 (v/v);
separating the neutral polysaccharide fraction from the first supernatant to produce the neutral polysaccharide fraction; and
optionally further purifying the neutral polysaccharide fraction by repeating the previous three steps except using the neutral polysaccharide fraction.

The Applicants have also obtained an acidic polysaccharide rich fraction (PGB-003), from the intermediate polysaccharide. Accordingly, the process further comprises preparing an acidic polysaccharide rich fraction. Therefore, the process further comprises:
adding C₁₋₃ alkylOH to the first supernatant to produce an acidic polysaccharide rich fraction and a second supernatant wherein the ratio of the 3 alkylOH to first supernatant is about 0.5 (v/v) to about 1 (v/v) to about 8 (v/v) to about 1 (v/v), and
separating the acidic polysaccharide rich fraction from the second supernatant to produce the acidic polysaccharide rich fraction.

Therefore, the present application also includes a process of preparing an acidic polysaccharide rich fraction, the process comprising:
providing an intermediate polysaccharide fraction produced by the process described above;
dissolving the intermediate polysaccharide fraction in water to produce an intermediate polysaccharide fraction solution wherein the intermediate polysaccharide fraction solution is about 1% (w/w) to about 10% (w/w),
adding C₁₋₃ alkylOH to the intermediate polysaccharide fraction solution to produce a neutral polysaccharide fraction and a first supernatant, wherein the ratio of the C₁₋₃ alkylOH to the intermediate polysaccharide fraction solution is about 1(v/v) to about 1 (v/v) to about 0.5 (v/v) to about 1 (v/v),
separating the first supernatant from the neutral polysaccharide fraction;
adding C₁₋₃ alkylOH to the first supernatant to produce an acidic polysaccharide rich fraction and a second supernatant, wherein the ratio of the C₁₋₃ alkylOH to first supernatant is about 0.5 (v/v) to about 1 (v/v) to about 8 (v/v) to about 1 (v/v); and
separating the acidic polysaccharide rich fraction from the second supernatant to produce the acidic polysaccharide rich fraction.

In an embodiment, the intermediate polysaccharide fraction solution is about 2% (w/w) to about 10% (w/w), about 3% (w/w) to about 10% (w/w), about 4% (w/w) to about 10% (w/w), about 5% (w/w) to about 10% (w/w), about 4% (w/w) to about 9% (w/w), about 4% (w/w) to about 8% (w/w), about 4% (w/w) to about 7% (w/w), about 4% (w/w) to about 6% (w/w), about 2% (w/w), about 3% (w/w), about 4% (w/w), about 5% (w/w), or about 6% (w/w). In an embodiment, the intermediate polysaccharide fraction solution is about 5% (w/w).

In an embodiment, the ratio of C₁₋₃ alkylOH to the intermediate polysaccharide fraction solution is about 1 (v/v) to about 1 (v/v) to about 0.5 (v/v) to about 1 (v/v). In an embodiment, the ratio of C₁₋₃ alkylOH to the intermediate polysaccharide fraction solution is about 1 (v/v) to about 1 (v/v) to about 0.5 (v/v) to about 1 (v/v) or about 0.7 (v/v) to about 1 (v/v) to about 0.5 (v/v) to about 1 (v/v). In an embodiment, the ratio of C₁₋₃ alkylOH to the intermediate polysaccharide fraction solution in the step of adding C₁₋₃ alkylOH to the intermediate polysaccharide fraction solution is about 0.5 (v/v) to about 1 (v/v).

In an embodiment, the step of producing the neutral polysaccharide rich fraction and the first supernatant is performed over more than about 1 hour, more than about 2 hours, more than about 3 hours or more than about 4 hours, more than about 5 hours or more than about 6 hours. In an embodiment, the step of producing the neutral polysaccharide rich fraction and the first supernatant is performed over more than about 3 hours. In an embodiment, the step of producing the neutral polysaccharide rich fraction and the first supernatant is performed over about 3 hours after the adding the C₁₋₃ alkylOH to the intermediate polysaccharide fraction solution.

In an embodiment, the C₁₋₃ alkylOH is added to the first supernatant in a ratio of 0.5 to about 1 (v/v) to about 6 to about 1 (v/v) of the C₁₋₃ alkylOH to the first supernatant. In an embodiment, the C₁₋₃ alkylOH is added to the first supernatant in a ratio of 1 to about 1 (v/v) to about 4 to about 1 (v/v) of the C₁₋₃ alkylOH to the first supernatant. In an embodiment, the C₁₋₃ alkylOH is added to the first supernatant in a ratio of about 2 to about 1 (v/v) of C₁₋₃ alkylOH to first supernatant.

It would be appreciated by a person skilled in the art that the acidic polysaccharide rich fraction can be separated from the second supernatant by any method of separating known in the art. In an embodiment, the separating the aqueous fraction is by filtering, decanting or centrifugation. In an embodiment, the separating is by centrifugation.

It would be appreciated by a person skilled in the art that a length time sufficient to produce the acidic polysaccharide rich fraction and the second supernatant is allowed after the C₁₋₃ alkylOH is added to first supernatant. In an embodiment, the step of producing the acidic polysaccharide rich fraction and the second supernatant is performed over more than about 1 hour, more than about 2 hours, more than about 3 hours, more than about 4 hours, more than about 5 hours or more than about 6 hours. In an embodiment, the step of producing the acidic polysaccharide rich fraction and the second supernatant is performed over more than about 3 hours. In an embodiment, the step of producing the acidic polysaccharide rich fraction and the second supernatant is performed over about 1 hour to about 5 hours, about 2 hours to about 3 hours, about 2 hours to about 4 hours, about 1 hour to about 4 hours or about 1 hour to about 3 hours. In an embodiment, the step of producing the acidic polysaccharide rich fraction and the second supernatant is performed over about 1 hour to about 3 hours. In an embodiment, the step of producing the acidic polysaccharide rich fraction and the second supernatant performed over about 3 hours.

In an embodiment, the acidic polysaccharide rich fraction is a solid, an oil, a wax or a gum. In an embodiment, the acidic polysaccharide rich fraction is a solid. In an embodiment, the acidic polysaccharide rich fraction is a solid, and the solid is a powder.

In an embodiment, the C₁₋₃ alkylOH in the steps described above s independently selected from methanol, ethanol, propanol and isopropanol. In an embodiment, the C₁₋₃ alkylOH in steps described above is independently selected from methanol and ethanol. In an embodiment, the C₁₋₃ alkylOH in the steps described above is ethanol.

The present application includes a process of preparing an acidic polysaccharide rich fraction, the process comprising:
providing an intermediate polysaccharide fraction produced by the process described above;
dissolving the intermediate polysaccharide fraction in water to produce an intermediate polysaccharide fraction solution wherein the intermediate polysaccharide fraction solution is about 1% (w/w) to about 10% (w/w),
adding ethanol to the intermediate polysaccharide fraction solution to produce a neutral polysaccharide fraction and a first supernatant, wherein the ratio of ethanol to the intermediate polysaccharide rich fraction solution is about 1 (v/v) to about 1 (v/v) to about 0.5 (v/v) to about 1 (v/v),
separating the first supernatant from the neutral polysaccharide rich fraction;
adding ethanol to the first supernatant to produce an acidic polysaccharide rich fraction and a second supernatant, wherein the ratio of the ethanol to first supernatant is about 0.5 (v/v) to about 1 (v/v) to about 8 (v/v) to about 1 (v/v), and
separating the acidic polysaccharide rich fraction from the second supernatant to produce the acidic polysaccharide rich fraction.

The present application includes a process of preparing an acidic polysaccharide rich fraction from a root portion of an American ginseng (Panax quinquefolius) plant, the process comprising:
extracting the root portion from a root portion of an American ginseng (Panax quinquefolius) plant with a solvent mixture comprising about 50% to about 95% (v/v) C₁₋₃ alkylOH and water at a temperature of about 10° C. to about 90° C. to produce a solvent mixture fraction and a first residue, wherein the ratio of the solvent mixture to the root portion is about 20 (v/w) to about 1 (v/w) to about 1 (v/w) to about 1 (v/w),
separating the first residue from the solvent mixture fraction;
extracting the first residue with water at a temperature of about 10° C. to about 100° C. to produce an aqueous fraction comprising the intermediate polysaccharide fraction and a second residue wherein the ratio of the water to the root portion is about 20 to about 1 (v/w) to about 1 to about 1 (v/w),
separating the aqueous fraction comprising the intermediate polysaccharide fraction from the second residue;
drying or concentrating the aqueous fraction to produce the intermediate polysaccharide fraction;
dissolving the intermediate polysaccharide fraction in water to produce an intermediate polysaccharide fraction solution, wherein the intermediate polysaccharide fraction solution is about 1% (w/w) to about 10% (w/w),
adding C₁₋₃ alkylOH to the intermediate polysaccharide fraction solution to produce a neutral polysaccharide fraction and a first supernatant wherein the ratio of the C₁₋₃ alkylOH to the intermediate polysaccharide fraction solution is about 1 (v/v) to about 1 (v/v) to about 0.5 (v/v) to about 1 (v/v),
separating the first supernatant from the neutral polysaccharide fraction;
adding C₁₋₃ alkylOH to the first supernatant to produce an acidic polysaccharide rich fraction and a second supernatant wherein the ratio of the 3 alkylOH to first supernatant is about 0.5 to about 1 (v/v) to about 8 to about 1 (v/v), and
separating the acidic polysaccharide rich fraction from the second supernatant to produce the acidic polysaccharide rich fraction.

The Applicants have further obtained a purified acidic polysaccharide fraction (PGB-007) from the acidic polysaccharide rich fraction. Accordingly, the process further comprises preparing an acidic polysaccharide fraction. Therefore, the process further comprises:
dissolving the acidic polysaccharide rich fraction in water to produce an acidic polysaccharide rich fraction solution wherein the acidic polysaccharide rich fraction solution is about 1% (w/w) to about 10% (w/w),
adding C₁₋₃ alkylOH to the acidic polysaccharide rich fraction solution to produce a precipitate and a third supernatant comprising the acidic polysaccharide fraction wherein the ratio of C₁₋₃ alkylOH to the acidic polysaccharide rich fraction solution about 1 (v/v) to about 1 (v/v) to about 0.5 (v/v) to about 1 (v/v),
separating the third supernatant comprising the acidic polysaccharide fraction from the precipitate; and
optionally, drying or concentrating the third supernatant to produce the acidic polysaccharide fraction.

Therefore, the present application further includes a process of preparing an acidic polysaccharide fraction (PGB-007), the process comprising:
providing an acidic polysaccharide rich fraction produced by the process described above;
dissolving the acidic polysaccharide rich fraction in water to produce an acidic polysaccharide rich fraction solution wherein the acidic polysaccharide rich fraction solution is about 1% (w/w) to about 10% (w/w),
adding C₁₋₃ alkylOH to the acidic polysaccharide rich fraction solution to produce a precipitate and a third supernatant comprising the acidic polysaccharide fraction wherein the ratio of C₁₋₃ alkylOH to the acidic polysaccharide rich fraction solution about 1 to about 1 (v/v) to about 0.5 to about 1 (v/v),
separating the third supernatant comprising the acidic polysaccharide fraction from the precipitate; and
optionally, drying or concentrating the third supernatant to produce the acidic polysaccharide fraction.

In an embodiment, the acidic polysaccharide rich fraction solution is about 2% to about 10% (w/w), about 3% to about 10% (w/w), about 4% to about 10% (w/w), about 5% to about 10% (w/w), about 4% to about 9% (w/w), about 4% to about 8% (w/w), about 4% to about 7% (w/w), about 4% to about 6% (w/w); about 2%, about 3% (w/w), about 4% (w/w), about 5% (w/w), or about 6% (w/w). In an embodiment, the intermediate polysaccharide fraction solution is about 5% (w/w).

In an embodiment, the ratio of C₁₋₃ alkylOH to the intermediate polysaccharide fraction solution is about 1 (v/v) to about 1 (v/v) to about 0.5 (v/v) to about 1 (v/v). In an embodiment, the ratio of C₁₋₃ alkylOH to the intermediate polysaccharide fraction solution is about 1 (v/v) to about 1 (v/v) to about 0.5 (v/v) to about 1 (v/v) or about 0.7 (v/v) to about 1 (v/v) to about 0.5 to about 1 (v/v) of the 3 alkylOH to the acidic polysaccharide rich fraction solution. In an embodiment, the ratio of the C₁₋₃ alkylOH to the polysaccharide rich fraction solution is about 0.5 to about 1 (v/v).

In an embodiment, the C₁₋₃ alkylOH is methanol, ethanol, propanol, or isopropanol. In an embodiment, the C₁₋₃ alkylOH is methanol or ethanol. In an embodiment, the C₁₋₃ alkylOH is ethanol

In an embodiment, the step of producing the precipitate and the third supernatant is performed over more than about 1 hour, more than about 2 hours, more than about 3 hours, more than about 4 hours, more than about 5 hours or more than about 6 hours. In an embodiment, the step of producing the precipitate and the third supernatant is performed over more than about 1 hour, more than about 2 hours, or more than about 3 hours. In an embodiment, the step of producing the precipitate and the third supernatant is performed over about 1 hour to about 5 hours, about 2 hours to about 3 hours, about 2 hours to about 4 hours, about 1 hour to about 4 hours or about 1 hour to about 3 hours. In an embodiment, the step of producing the precipitate and the third supernatant is performed over about 1 hour to about 3 hours. In an embodiment, the step of producing the precipitate and the third supernatant is performed over about 3 hours.

It would be appreciated by a person skilled in the art that each of the separating steps described above can be performed using any method known in the art. In an embodiment, the separating is by filtering, decanting or centrifugation. In an embodiment, the separating is by centrifugation.

It would be appreciated by a person skilled in the art that “optionally’, for example, as in “optionally, drying or concentrating the third supernatant to produce the acidic polysaccharide fraction (PGB-007), means that the third supernatant comprising the acidic polysaccharide fraction can be used as produced or the solvent from the third supernatant can be removed or concentrated to produce the acidic polysaccharide fraction.

In an embodiment, the drying or concentrating is by any method of solvent removal known in the art. In an embodiment, the drying or concentrating is by evaporation. In an embodiment, the evaporation is by distillation, open air evaporation, evaporation under vacuum, or rotoevapouration. In an embodiment, the evaporation is by rotoevapouration.

In an embodiment, the drying comprises concentrating the third supernatant comprising the acidic polysaccharide fraction followed by freeze drying or spray drying to produce the acidic polysaccharide fraction. In an embodiment, the drying comprises concentrating the aqueous fraction comprising the acidic polysaccharide fraction followed by freeze drying to produce the acidic polysaccharide fraction.

In an embodiment, the acidic polysaccharide rich fraction produced by the processes as described above can be used without further purification. In an embodiment, the acidic polysaccharide rich fraction is further purified as described above to produce purified acidic polysaccharide fraction.

In an embodiment, the acidic polysaccharide fraction produced after drying is a solid, an oil, a wax or a gum. In an embodiment, the acidic polysaccharide fraction is a solid. In an embodiment, the acidic polysaccharide fraction is a solid, and the solid is a powder.

In an embodiment, the process provides the acidic polysaccharide fraction in a purity of between about 80% to about 100%, about 85% to about 100%, about 90% to about 100%, about 95% to about 100%, about 95% to about 98% or about 85% (w/w) or more.

In an embodiment, the process provides the acidic polysaccharide fraction in an overall yield of about 10% to about 15% when the intermediate polysaccharide fraction is taken as the initial starting material.

The present application includes a process of preparing an acidic polysaccharide fraction, the process comprising
providing an acidic polysaccharide rich fraction produced by the process described above;
dissolving the acidic polysaccharide rich fraction in water to produce an acidic polysaccharide rich fraction solution wherein the acidic polysaccharide rich fraction solution is about 1% to about 10% (w/w),
adding ethanol to the acidic polysaccharide rich fraction solution to produce a precipitate and a third supernatant comprising the acidic polysaccharide fraction, wherein the ratio of ethanol to the acidic polysaccharide rich fraction solution about 1 (v/v) to about 1 (v/v) to about 0.5 (v/v) to about 1 (v/v),
separating the third supernatant comprising the acidic polysaccharide fraction from the precipitate; and
optionally, drying or concentrating the third supernatant to produce the acidic polysaccharide fraction.

Alternatively, the acidic polysaccharide rich fraction is purified using column chromatography to produce the acidic polysaccharide fraction. Accordingly, the process further comprises:
fractionating the acidic polysaccharide rich fraction from a chromatographic column comprising a diethylaminoethyl (DEAE) anion exchange resin to produce an acidic polysaccharide eluent fraction, wherein the fractionating comprises
(i) dissolving the acidic polysaccharide rich fraction in water to produce an acidic polysaccharide rich fraction solution;
(ii) loading the acidic polysaccharide rich fraction solution on the chromatographic column comprising the anion exchange resin; and
(iii) eluting the acidic polysaccharide rich eluent fraction from the anion exchange resin using an ammonium acetate buffer to produce the acidic polysaccharide eluent fraction, wherein the eluting comprises a first step wherein the buffer is about 10 mM to about 100 mM of ammonium acetate, and a second step wherein the buffer comprises about 0.5M to about 1.5M of ammonium acetate; and
optionally, drying or concentrating the acidic polysaccharide eluent fraction to produce the acidic polysaccharide fraction.

Therefore, the present application also includes a process of preparing an acidic polysaccharide fraction, the process comprising:
providing an acidic polysaccharide rich fraction produced by a process described above;
fractionating the acidic polysaccharide rich fraction from a chromatographic column comprising a diethylaminoethyl (DEAE) anion exchange resin to produce an acidic polysaccharide eluent fraction, wherein the fractionating comprises
(i) dissolving the acidic polysaccharide rich fraction in water to produce an acidic polysaccharide rich fraction solution;
(ii) loading the acidic polysaccharide rich fraction solution on the chromatographic column comprising the anion exchange resin; and
(iii) eluting the acidic polysaccharide rich eluent fraction from the anion exchange resin using an ammonium acetate buffer to produce the acidic polysaccharide eluent fraction, wherein the eluting comprises a first step wherein the buffer is about 10 mM to about 100 mM of ammonium acetate, and a second step wherein the buffer comprises about 0.5M to about 1.5M of ammonium acetate; and
optionally, drying or concentrating the acidic polysaccharide eluent fraction to produce the acidic polysaccharide fraction.

In an embodiment, the chromatographic column is for use with a high pressure liquid chromatography (HPLC) system. Accordingly, in an embodiment, the chromatographic column is an anion exchange HPLC column. In an embodiment, the (HPLC) system is an Agilent® 1200 series preparative HPLC system.

In an embodiment, the resin comprises cross-linked polymethacrylate.

In an embodiment, the buffer in the first step of the eluting is about 10 mM to about 75 mM of ammonium acetate, about 10 mM to about 50 mM of ammonium acetate, about 10 mM to about 30 mM of ammonium acetate, or about 10 mM to about 20 mM of ammonium acetate. In an embodiment, the buffer in the first step of the eluting is about 20 mM ammonium acetate.

In an embodiment, the buffer in the second step of the eluting is about 0.75 M to about 1.25M ammonium acetate. In an embodiment, the buffer in the second step of the eluting is about 1M of ammonium acetate.

In an embodiment, the chromatographic column has a bed dimension of about 150 mm×about 21.5 mm and a bed volume of about 54.5 mL.

In an embodiment, the chromatographic column comprising an anion exchange resin is a TSK® gel 13 μm DEAE-5PW column.

In an embodiment, the acidic polysaccharide eluent fraction is the fraction corresponding to a retention time of about 42 minutes to about 100 minutes when the fractionating is performed using a TSK® gel 13 μm DEAE-5PW column with a flow rate of about 3 ml/min and an injection volume of about 5 ml.

The processes for performing anion exchange chromatography are known to those skilled in the art following manufacturer's recommendations as to flow rate, sample volume and temperatures at which the procedure should be performed.

It would be appreciated by a person skilled in the art that “optionally’ in the step of drying or concentrating the acidic polysaccharide eluent fraction means that the eluent fraction comprising the acidic polysaccharide fraction can be used as produced or the solvent from the eluent fraction can be removed to produce the acidic polysaccharide fraction.

In an embodiment, the process provides the acidic polysaccharide fraction in a purity of between about 80% to about 100%, about 85% to about 100%, about 90% to about 100%, about 95% to about 100%, about 95% to about 98% or about 95% (w/w) or more.

In an embodiment, the process provides the acidic polysaccharide fraction is obtained in an overall yield of about 10% to about 15% when the intermediate polysaccharide fraction is taken as the initial starting material.

In an embodiment, the step of drying or concentrating is by evaporation. In an embodiment, the evaporation is by distillation, open air evaporation, evaporation under vacuum, or rotoevapouration. In an embodiment, the evaporation is by rotoevapouration.

In an embodiment, the step of drying comprises concentrating the acidic polysaccharide eluent fraction to produce the acidic polysaccharide fraction followed by freeze drying or spray drying to produce polysaccharide fraction. In an embodiment, the drying comprises concentrating the acidic polysaccharide eluent fraction comprising the acidic polysaccharide fraction followed by freeze drying to produce polysaccharide fraction.

In an embodiment, the C₁₋₃ alkylOH used in each step of the processes of preparing the fractions of the application described above is the same or may be different.

### III Extracts and Fractions of the Application

The Applicants have obtained intermediate polysaccharide fraction, and purified neutral polysaccharide fraction (PBG-005), acidic polysaccharide rich fraction and acidic polysaccharide fraction from the root portion of an American ginseng (Panax quinquefolius) plant.

Accordingly, the present application includes an intermediate polysaccharide fraction (PBG-002) produced by the process described above.

In an embodiment, the present application includes an intermediate polysaccharide fraction (PBG-002) having a carbohydrate content comprising about 0.5 to about 4 mole % rhamnose (Rha), about 8 to about 20 mole % galacturonic acid (GalA), about 60 to about 80 mole % glucose (Glc), about 3 to about 10 mole % galactose (Gal) and about 3 to about 10 mole % arabinose (Ara). In an embodiment, the present application includes an intermediate polysaccharide fraction (PBG-002) having a carbohydrate content comprising about 0.5 to about 4 mole % rhamnose (Rha), about 13 to about 17 mole % galacturonic acid (GalA), about 70 to about 74 mole % glucose (Glc), about 3 to about 7 mole % galactose (Gal) and about 3 to about 7 mole % arabinose (Ara).

In an embodiment, the present application includes an intermediate polysaccharide fraction having a carbohydrate content comprising about 1 to about 2 mole % rhamnose (Rha), about 14 to about 16 mole % galacturonic acid (GalA), about 71 to about 73.0 mole % glucose (Glc), about 4 to about 6 mole % galactose (Gal) and about 4 to about 6 mole % arabinose (Ara).

In an embodiment, the present application includes an intermediate polysaccharide fraction having a carbohydrate content comprising about 0.9 to about 1.7 mole % rhamnose (Rha), about 14.6 to about 15.5 mole % galacturonic acid (GalA), about 72.0 to about 73.0 mole % glucose (Glc), about 4.9 to about 5.7 mole % galactose (Gal) and about 5.0 to about 5.6 mole % arabinose (Ara).

In an embodiment, the present application includes an intermediate polysaccharide fraction having a carbohydrate content comprising about 1.3 mole % rhamnose (Rha), about 15.1 mole % galacturonic acid (GalA), about 72.4 mole % glucose (Glc), 5.3 mole % galactose (Gal) and about 5.6 mole % arabinose.

In an embodiment, the present application also includes a neutral polysaccharide fraction (PGB-005) produced by the process described above.

The present application also includes a neutral polysaccharide fraction having a carbohydrate content comprising about 0.1 to about 5 mole % rhamnose (Rha), about 2 to about 10 mole % galacturonic acid (GalA), about 80 to about 95 mole % glucose (Glc), about 1 to about 5 mole % galactose (Gal) and about 1 to about 5 mole % arabinose (Ara). The present application also includes a neutral polysaccharide fraction having a carbohydrate content comprising about 0.1 to about 3 mole % rhamnose (Rha), about 3 to about 7 mole % galacturonic acid (GalA), about 87 to about 92 mole % glucose (Glc), about 1 to about 4 mole % galactose (Gal) and about 1 to about 5 mole % arabinose (Ara).

The present application also includes a neutral polysaccharide fraction having a carbohydrate content comprising about 0.1 to about 2 mole % rhamnose (Rha), about 5.0 to about 6.0 mole % galacturonic acid (GalA), about 89 to about 92 mole % glucose (Glc), about 1 to about 3 mole % galactose (Gal) and about 1 to about 3 mole % arabinose (Ara).

The present application also includes a neutral polysaccharide fraction having a carbohydrate content comprising about 0.1 to about 1.1 mole % rhamnose (Rha), about 5.0 to about 6.0 mole % galacturonic acid (GalA), about 89.3 to about 91.2 mole % glucose (Glc), about 1.4 to about 2.3 mole % galactose (Gal) and about 1.6 to about 2.4 mole % arabinose (Ara).

The present application also includes a neutral polysaccharide fraction having a carbohydrate content comprising about 0.6 mole % rhamnose (Rha), about 5.6 mole % galacturonic acid (GalA), about 89.8 mole % glucose (Glc), about 1.9 mole % galactose (Gal) and about 2.0 mole % arabinose (Ara).

The present application also includes a neutral polysaccharide fraction comprising a terminally linked arabinofuranosyl residue (t-Araf), a terminally linked glucopyranosyl residue (t-Glcp), a 4-linked mannopyranosyl residue (4-Manp), a 4-linked glucopyranosyl residue (4-Glcp), and a 4,6-linked glucopyranosyl residue (4, 6-Glop).

The present application also includes a neutral polysaccharide fraction comprising about 3 to about 7% terminally linked arabinofuranosyl residue (t-Araf), about 3 to about 7 wt % terminally linked glucopyranosyl residue (t-Glcp), about 0.1 to about 4 wt % 4-linked mannopyranosyl residue (4-Manp), about 90 to about 94 wt % 4-linked glucopyranosyl residue (4-Glcp), and about 0.1 to 3 wt % 4,6-linked glucopyranosyl residue (4, 6-Glcp).

The present application also includes a neutral polysaccharide fraction comprising about 4.5 to about 5.5% terminally linked arabinofuranosyl residue (t-Araf), about 4.9 to about 5.9 wt % terminally linked glucopyranosyl residue (t-Glcp), about 0.2 to about 1.2 wt % 4-linked mannopyranosyl residue (4-Manp), about 91.1 to about 92.1 wt % 4-linked glucopyranosyl residue (4-Glcp), and about 1.1 to about 2.1 wt % 4,6-linked glucopyranosyl residue (4, 6-Glcp).

The present application also includes a neutral polysaccharide fraction comprising about 5.0% terminally linked arabinofuranosyl residue (t-Araf), about 5.4 wt % terminally linked glucopyranosyl residue (t-Glcp), about 0.8 wt % 4-linked mannopyranosyl residue (4-Manp), about 91.6 wt % 4-linked glucopyranosyl residue (4-Glcp), and about 1.6 wt % 4,6-linked glucopyranosyl residue (4, 6-Glcp).

In an embodiment, the present application includes an acidic polysaccharide rich fraction (PGB-003) produced by a process described above.

In an embodiment, the present application includes an acidic polysaccharide fraction (PGB-007) produced by a process described above.

The present application includes an acidic polysaccharide fraction having a carbohydrate content comprising about 1 to about 8 mole % rhamnose (Rha), about 40 to about 50 mole % galacturonic acid (GalA), about 8 to about 16 mole % glucose (Glc), about 14 to about 25 mole % galactose (Gal) and about 15 to about 25 mole % arabinose (Ara). The present application includes an acidic polysaccharide fraction having a carbohydrate content comprising about 2 to about 6 mole % rhamnose (Rha), about 43 to about 47 mole % galacturonic acid (GalA), about 10 to about 14 mole % glucose (Glc), about 18 to about 22 mole % galactose (Gal) and about 16 to about 20 mole % arabinose (Ara).

The present application includes an acidic polysaccharide fraction having a carbohydrate content comprising about 3 to about 5 mole % rhamnose (Rha), about 45 to about 47 mole % galacturonic acid (GalA), about 11 to about 13 mole % glucose (Glc), about 19 to about 21 mole % galactose (Gal) and about 17 to about 19 mole % arabinose (Ara).

The present application includes an acidic polysaccharide fraction having a carbohydrate content comprising about 3.6 to about 4.4 mole % rhamnose (Rha), about 45.1 to about 46.0 mole % galacturonic acid (GalA), about 11.8 to about 12.6 mole % glucose (Glc), about 19.2 to about 20.3 mole % galactose (Gal) and about 17.7 to about 18.7 mole % arabinose (Ara).

The present application includes an acidic polysaccharide fraction having a carbohydrate content comprising about 4.0 mole % rhamnose (Rha), about 45.6 mole % galacturonic acid (GalA), about 12.2 mole % glucose (Glc), about 19.8 mole % galactose (Gal) and about 18.2 mole % arabinose (Ara).

The present application also includes an acidic polysaccharide fraction comprising a terminally linked rhaminopyranosyl residue (t-Rha), a terminally linked arabinofuranosyl residue (t-Araf), a 2-linked rhaminopyranosyl residue (2-Rha), a terminally linked glucopyranosyl residue (t-Glcp), a terminally linked galactopyranosyl residue and terminally linked galacturonic acid pyranosyl residue (t-Galp & t-Gal A), a 4-linked arabinopyranosyl residue or 5-linked arabinofuranosyl residue (4-Arap or 5-Araf), a 2,4-linked rhaminopyranosyl residue (2,4-Rha), a 3-linked galactopyranosyl residue (3-Galp), a 4-linked galactopyranosyl residue and 4-linked galacturonic acid pyranosyl residue (4-Galp & 4-Gal A), a 4-linked glucopyranosyl residue (4-Glcp), a 2,4-linked galactopyranosyl residue (2,4-Galp), and a 3,6-linked galactopyranosyl residue (3,6-Galp).

The present application also includes as acidic polysaccharide fraction comprising about 2 to about 5 wt % terminally linked rhaminopyranosyl residue (t-Rha), about 3 to about 6 wt % terminally linked arabinofuranosyl residue (t-Araf), about 1 to about 4 wt % 2-linked rhaminopyranosyl residue (2-Rha), about 3 to about 6 wt % terminally linked glucopyranosyl residue (t-Glcp), about 12 to about 15 wt % terminally linked galactopyranosyl residue and terminally linked galacturonic acid pyranosyl residue (t-Galp & t-Gal A), about 4 to about 8 wt % 4-linked arabinopyranosyl residue or 5-linked arabinofuranosyl residue (4-Arap or 5-Araf), about 1 to about 4 wt % 2,4-linked rhaminopyranosyl residue (2,4-Rha), about 1 to about 4 wt % 3-linked galactopyranosyl residue (3-Galp), about 31 to about 35 wt % 4-linked galactopyranosyl residue and 4-linked galacturonic acid pyranosyl residue (4-Galp & 4-Gal A), about 22 to about 26 wt % 4-linked glucopyranosyl residue (4-Glcp), about 0.5 to about 3 wt % 2,4-linked galactopyranosyl residue (2,4-Galp), and about 0.5 to about 3 wt % 3,6-linked galactopyranosyl residue (3,6-Galp).

The present application also includes as acidic polysaccharide fraction comprising about 3.3 to about 4.2 wt % terminally linked rhaminopyranosyl residue (t-Rha), about 4.1 to about 5.0 wt % terminally linked arabinofuranosyl residue (t-Araf), about 2.1 to about 3.1 wt % 2-linked rhaminopyranosyl residue (2-Rha), about 4.4 to about 5.4 wt % terminally linked glucopyranosyl residue (t-Glcp), about 13.2 to about 14.2 wt % terminally linked galactopyranosyl residue and terminally linked galacturonic acid pyranosyl residue (t-Galp & t-Gal A), about 6.0 to abut 7.0 wt % 4-linked arabinopyranosyl residue or 5-linked arabinofuranosyl residue (4-Arap or 5-Araf), about 1.9 to about 2.9 wt % 2,4-linked rhaminopyranosyl residue (2,4-Rha), about 2.0 to about 3.0 wt % 3-linked galactopyranosyl residue (3-Galp), about 32.5 to about 33.3 wt % 4-linked galactopyranosyl residue and 4-linked galacturonic acid pyranosyl residue (4-Galp & 4-Gal A), about 23.6 to about 24.4 wt % 4-linked glucopyranosyl residue (4-Glcp), about 0.8 to about 1.8 wt % 2,4-linked galactopyranosyl residue (2,4-Galp), and about 0.8 to about 1.6 wt % 3,6-linked galactopyranosyl residue (3,6-Galp).

The present application also includes as acidic polysaccharide fraction comprising about 3.7 wt % terminally linked rhaminopyranosyl residue (t-Rha), about 4.5 wt % terminally linked arabinofuranosyl residue (t-Araf), about 2.6 wt % 2-linked rhaminopyranosyl residue (2-Rha), about 4.9 wt % terminally linked glucopyranosyl residue (t-Glcp), about 13.7 wt % terminally linked galactopyranosyl residue and terminally linked galacturonic acid pyranosyl residue (t-Galp & t-Gal A), about 6.4 wt % 4-linked arabinopyranosyl residue or 5-linked arabinofuranosyl residue (4-Arap or 5-Araf), about 2.4 wt % 2,4-linked rhaminopyranosyl residue (2,4-Rha), about 2.5 wt % 3-linked galactopyranosyl residue (3-Galp), about 32.9 wt % 4-linked galactopyranosyl residue and 4-linked galacturonic acid pyranosyl residue (4-Galp & 4-Gal A), about 24.0 wt % 4-linked glucopyranosyl residue (4-Glcp), about 1.3 wt % 2,4-linked galactopyranosyl residue (2,4-Galp), and about 1.2 wt % 3,6-linked galactopyranosyl residue (3,6-Galp).

In an embodiment, the present application also includes salts and/or solvates of the intermediate polysaccharide fraction, neutral polysaccharide fraction, acidic polysaccharide rich fraction and/or acidic polysaccharide fraction.

In an embodiment the salt is an acid addition salt or a base addition salt.

The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic fractions of the application that form an acid addition salt include, for example, fractions of the application comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of fractions of the application of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic fractions of the application that form a basic addition salt include, for example, fractions of the application comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a fraction is not hydrolyzed. The selection criteria for the appropriate salt will be known to one skilled in the art.

In an embodiment the salt is a base addition salt.

The formation of a desired compound salt is achieved using standard techniques. For example, the neutral compound is treated with an acid or base in a suitable solvent and the formed salt is isolated by filtration, extraction or any other suitable method.

Solvates of fractions of the application include, for example, those made with solvents that are pharmaceutically acceptable. Examples of such solvents include water (resulting solvate is called a hydrate) and ethanol and the like. The selection of suitable conditions to form a particular solvate can be made by a person skilled in the art.

In embodiments of the present application, the one or more fractions of the application described herein may have at least one asymmetric center. Where fractions of the application possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The one or more fractions of the application of the present application may exist in varying polymorphic forms and it is contemplated that any polymorphs, or mixtures thereof, which form are included within the scope of the present application.

It is intended that any tautomeric forms which the fractions of the application form, as well as mixtures thereof, are included within the scope of the present application.

### IV. Compositions of the Application

The one or more fractions (for example, intermediate polysaccharide fraction (PBG-002), neutral polysaccharide fraction (PBG-005), acidic polysaccharide rich fraction and acidic polysaccharide fraction) of the present application are suitably formulated in a conventional manner into compositions using one or more carriers. Accordingly, the present application also includes a composition comprising one or more fractions of the application and a carrier. The one or more fractions of the application are suitably formulated into pharmaceutical compositions for administration to subjects in a biologically compatible form suitable for administration in vivo. Accordingly, the present application further includes a pharmaceutical composition comprising one or more fractions of the application and a pharmaceutically acceptable carrier. In an embodiment, the one or more fractions of the application are suitably formulated into nutraceutical compositions for administration to subjects in a biologically compatible form suitable for administration in vivo. Accordingly, the present application further includes a nutraceutical composition comprising one or more fractions of the application and a carrier. In an embodiment, the carrier is a pharmaceutically acceptable carrier.

The one or more fractions of the application may be administered to a subject in a variety of forms depending on the selected route of administration, as will be understood by those skilled in the art. One ore more fractions of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

One or more fractions of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the one or more fractions may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g., liposomes or those wherein the one or more fractions is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the one or more fractions of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the one or more fractions of the application and use the lyophilizates obtained, for example, for the preparation of products for injection.

One or more fractions of the application may also be administered parenterally. Solutions of one or more fractions of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the one or more fractions of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The one or more fractions of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the one or more fractions of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the one or more fractions of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the one or more fractions of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, P A, 1980, pp. 1530-1533 for further discussion of suppository dosage forms.

One or more fractions of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, fractions of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In an embodiment, one or more fractions of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

The one or more fractions of the application including pharmaceutically acceptable salts, and solvates thereof are suitably used on their own but will generally be administered in the form of a pharmaceutical composition in which the one or more fractions of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient (one or more fractions of the application), and from about 1 wt % to about 99.95 wt % or about 30 wt % to about 99.90 wt % of a pharmaceutically acceptable carrier, all percentages by weight being based on the total composition.

In an embodiment, one or more fractions of the application is administered with another therapeutic agent simultaneously or sequentially in separate unit dosage forms or together in a single unit dosage form. Accordingly, the present application provides a single unit dosage form comprising one or more fractions of the application, additional therapeutic agent, and a pharmaceutically acceptable carrier.

The one or more fractions of the application may be used alone or in combination with another known agent useful for treating or preventing a viral infection.

It is an embodiment that another known agent useful for treating or preventing a viral infection are administered or used according to treatment protocol that is known the other known agents.

### IV. Methods and Uses of the Application

The fractions of the application have been shown to activate Toll-like receptor (TLR) activity and therefore shown to be TLR agonists. Therefore, the fractions of the application have been shown to be immunological agents, for example, immunostimulators, which, for example, activate the innate and adaptive immune system, in particular, the innate immune system. The fractions of the application also been shown to confer protection against viral infection, in both in vitro and in vivo models. Therefore, the fractions of the application have been shown to have antiviral activity.

Accordingly, the present application includes a method for activating Toll-like receptor (TLR) function in a cell, either in a biological sample or in a patient, comprising administering an effective amount of one or more fractions of the application to the cell. The application also includes a use of one or more fractions of the application for activating Toll-like receptor (TLR) function in a cell as well as a use of one or more fractions of the application for the preparation of a medicament for activating Toll-like receptor (TLR) function in a cell. The application further includes one or more fractions of the application for use in activating Toll-like receptor (TLR) function in a cell.

The fractions of the application have also been shown to stimulate the production of chemokines, in particular interleukin-8 (IL-8)

Accordingly, the present application also includes a method for stimulating the production of one or more chemokines in a cell, either in a biological sample or in a patient, comprising administering an effective amount of one or more fractions of the application to the cell. The application also includes a use of one or more fractions of the application for stimulating the production of one or more chemokines in a cell as well as a use of one or more fractions of the application for the preparation of a medicament for stimulating the production of one or more chemokines. The application further includes one or more fractions of the application for use in stimulating the production of one or more chemokines in a cell.

In an embodiment, the one or more chemokines are selected from IL-8, IL-2, IL-1, IL-6, INF-gamma, TNF-alpha and interferon-γ. In an embodiment, the one or more chemokines is IL-8.

As the fractions of the application have been shown to be capable of activating Toll-like receptor (TLR) function protein activity and stimulating the production of one or more chemokines, the fractions of the application are useful for treating diseases, disorders or conditions by activating Toll-like receptor (TLR) function or by stimulating the production of one or more chemokines. Therefore, the fractions of the present application are useful as medicaments. In an embodiment, the fractions of the present application are useful as nutraceuticals or a functional food.

The present application also includes a method of treating a disease, disorder or condition by activation of TLR function comprising administering a therapeutically effective amount of one or more fractions of the application to a subject in need thereof.

Accordingly, the present application also includes a use of one or more fractions of the application for treatment of a disease, disorder or condition by activation of TLR function as well as a use of one or more fractions of the application for the preparation of a medicament for treatment of a disease, disorder or condition by activation of TLR function. The application further includes one or more fractions of the application for use in treating a disease, disorder or condition by activation of TLR function.

In an embodiment, the TLR is TLR2 or TLR4. In an embodiment, the TLR is a dimer. In an embodiment, the TLR is a homodimer.

The present application also includes a method of treating a disease, disorder or condition by a stimulating the production of one or more chemokines comprising administering a therapeutically effective amount of one or more fractions of the application to a subject in need thereof.

The present application also includes a use of one or more fractions of the application for treatment of a disease, disorder or condition by stimulating the production of one or more chemokines as well as a use of one or more fractions of the application for the preparation of a medicament for treatment of a disease, disorder or condition by stimulating the production of one or more chemokines. The application further includes one or more fractions of the application for use in treating a disease, disorder or condition by stimulating the production of one or more chemokines.

Toll-like receptors (TLR) are known to participate in the activation or the innate and adaptive immune response. In particular, they are known to be responsible for mounting an adaptive and innate response.

Accordingly, the present application also includes a method for activating an innate and/or subsequent adaptive response in a cell, either in a biological sample or in a patient, comprising administering an effective amount of one or more fractions of the application to the cell. The application also includes a use of one or more fractions of the application for activating an innate and/or adaptive innate response in a cell as well as a use of one or more fractions of the application for the preparation of a medicament for activating an innate an/or adaptive innate response in a cell. The application further includes one or more fractions of the application for use in activating an innate and/or adaptive innate response in a cell in a cell.

The present application also includes a method of treating a disease, disorder or condition associated with activating an innate and/or adaptive innate response comprising administering a therapeutically effective amount of one or more fractions of the application to a subject in need thereof.

The present application also includes a use of one or more fractions of the application for treatment of a disease, disorder or condition associated with activating an innate and/or adaptive innate response as well as a use of one or more fractions of the application for the preparation of a medicament for treatment of a disease, disorder or condition associated with activating an innate and/or adaptive innate response. The application further includes one or more fractions of the application for use in treating a disease, disorder or condition by associated with activating an innate and/or adaptive innate response.

In an embodiment, the disease, disorder or condition treatable by activation of TLR function, by stimulation of the production of one or more chemokines and/or associated with activating an innate and/or adaptive innate response is a viral infection, microbial infection or cancer. Accordingly, the present application also includes a method of treating a viral infection, a microbial infection or a cancer comprising administering a therapeutically effective amount of one or more fractions of the application to a subject in need thereof.

The fractions of the application have been shown to have antiviral activity against viral infections, for example, against influenza virus infection and herpes simplex viral (HSV) infection, such as HSV-1 and HSV-2.

Accordingly, the present application also includes a method of treating or preventing a viral infection comprising administering a therapeutically effective amount of one or more fractions of the application to a subject in need thereof.

The present application also includes a use of one or more fractions of the application for treatment or prevention of a viral infection as well as a use of one or more fractions of the application for the preparation of a medicament for treatment or prevention of a viral infection. The application further includes one or more fractions of the application for use in treating or preventing a viral infection.

In an embodiment, the viral infection is an influenza virus infection, a herpesvirus infection, a human immunodeficiency virus (HIV) infection, a respiratory syncytial virus (RSV) infection, various cold and flu viruses or a coronavirus infection.

In an embodiment, the influenza virus infection is an infection of at least one influenza virus. In an embodiment, the influenza virus is an influenza A virus or influenza B virus. In an embodiment, the influenza A virus is a hemagglutinin (“H”) or a neuraminidase (“N”) subtype. In an embodiment, the influenza A virus subtype is selected from influenza A virus subtype H1N1, influenza A virus subtype H1N2, influenza A virus subtype H2N2, influenza A virus subtype H2N3, influenza A virus subtype H3N1, influenza A virus subtype H3N2, influenza A virus subtype H3N8, influenza A virus subtype H5N1, influenza A virus subtype H5N2, influenza A virus subtype H5N3, influenza A virus subtype H5N6, influenza A virus subtype H5N8, influenza A virus subtype H5N9, influenza A virus subtype H6N1, influenza A virus subtype H6N2, influenza A virus subtype H7N1, influenza A virus subtype H7N2, influenza A virus subtype H7N3, influenza A virus subtype H7N4, influenza A virus subtype H7N7, influenza A virus subtype H7N9, influenza A virus subtype H9N2 and influenza A virus subtype H10N7. In an embodiment, the influenza A virus subtype is influenza A virus subtype H5N1.

In an embodiment, the viral infection is an influenza A virus subtype H1N1 infection.

In an embodiment, the influenza A virus is an influenza A virus variant. In an embodiment, the influenza A virus variant is from avian influenza virus, human influenza virus, swine influenza virus, equine influenza virus, bat influenza virus, feline influenza virus and canine influenza virus. In an embodiment, the influenza A virus is human influenza virus.

In an embodiment, the herpesvirus infection is an infection of at least one herpesvirus infection. In an embodiment, the herpesvirus infection is selected from a herpes simplex virus 1 (HSV-1) infection, a herpes simplex virus (HSV-2), a varicella-zoster virus (VZV) infection, an Epstein-Barr virus (EBV) infection, a human cytomegalovirus (HCMV) infection, a human herpesvirus 6A (HHV-6a) infection, a human herpesvirus 6A (HHV-6B) infection, a human herpesvirus 7 (HHV-7) human herpesvirus 6A, and a Kaposi's sarcoma-associated herpesvirus (KSHV) infection.

In an embodiment, the herpesvirus infection is selected from a herpes simplex virus 1 (HSV-1) infection and a herpes simplex virus (HSV-2) infection.

In an embodiment, the human immunodeficiency virus (HIV) infection is a HIV-1 infection or a HIV-2 infection.

In an embodiment, the coronavirus infection is a severe acute respiratory syndrome coronavirus (SARS)-related coronavirus infection, or a Middle East respiratory syndrome-related coronavirus (MERS)-related coronavirus infection. Therefore, in an embodiment, the MERS-related coronavirus infection is a MERS-CoV or MERS-CoV variant infection.

In an embodiment, the SARS-related coronavirus infection is an infection of at least one SARS-related coronavirus. In an embodiment, the SARS-related coronavirus is SARS-CoV or SARS-CoV-2. In an embodiment, the SARS-related coronavirus is SARS-related coronavirus variant. In an embodiment, the SARS-related coronavirus variant is a SARS-CoV variant or a SARS-CoV-2 variant. Therefore, in an embodiment, the SARS-related coronavirus infection is a SARS-CoV-2 infection or a SARS-CoV-2 variant infection. In an embodiment, the SARS-related coronavirus infection is a SARS-CoV-2 infection

In an embodiment, the MERS-related coronavirus infection is an infection of at least one MERS-related coronavirus. In an embodiment, the MERS-related coronavirus is MERS-CoV. In an embodiment, the MERS-related coronavirus is MERS-related coronavirus variant. In an embodiment, the MERS-related coronavirus variant is a MERS-CoV variant.

By “variant”, it is meant, for example, a virus comprising one or more changes or mutations in the nucleotide sequence of the original virus genome.

In an embodiment, the viral infection is a primary viral infection or an opportunistic infection.

It would also be appreciated by the person skilled in the art that a disease, disorder or condition arising from a viral infection can be a primary disease, disorder or condition arising from a viral infection, or can be a secondary disease, disorder or condition arising from a viral infection as a result of a primary disease, disorder or condition arising from the viral infection.

In an embodiment, the cell is in vivo. In an embodiment, the cell is in vitro.

The present application also includes a method of treating a disease, disorder or condition that is treatable by activation of TLR function, by stimulation of the production of one or more chemokines and/or associated with activating an innate and/or adaptive innate response comprising administering a therapeutically effective amount of one or more fractions of the application in combination with another known agent useful for treatment of a disease, disorder or condition treatable by activation of TLR function, by stimulation of the production of one or more chemokines and/or associated with activating an innate and/or adaptive innate response to a subject in need thereof. The present application also includes a use of one or more fractions of the application in combination with another known agent useful for treatment of a disease, disorder or condition treatable by activation of TLR function, by stimulation of the production of one or more chemokines and/or associated with activating an innate and/or adaptive innate response for treatment of a disease, disorder or condition treatable by activation of TLR function, by stimulation of the production of one or more chemokines and/or associated with activating an innate and/or adaptive innate response, as well as a use of one or more fractions of the application in combination with another known agent useful for treatment of a disease, disorder or condition treatable by activation of TLR function, by stimulation of the production of one or more chemokines and/or associated with activating an innate and/or adaptive innate response for the preparation of a medicament for treatment of a disease, disorder or condition treatable by activation of TLR function, by stimulation of the production of one or more chemokines and/or associated with activating an innate and/or adaptive innate response. The application further includes one or more fractions of the application in combination with another known agent useful for treatment of a disease, disorder or condition treatable by activation of TLR function, by stimulation of the production of one or more chemokines and/or associated with activating an innate and/or adaptive innate response for use in treating a disease, disorder or condition treatable by activation of TLR function, by stimulation of the production of one or more chemokines and/or associated with activating an innate and/or adaptive innate response. In an embodiment, the disease, disorder or condition treatable by activation of TLR function, by stimulation of the production of one or more chemokines and/or associated with activating an innate and/or adaptive innate response is a viral or microbial infection such as influenza virus infection, a herpesvirus infection, a human immunodeficiency virus (HIV) infection, a respiratory syncytial virus (RSV) infection, or a coronavirus infection.

It would be appreciated by the person skilled in the art that a subject may be afflicted with two or more diseases, disorders or conditions arising from a viral infection at the same time.

In another embodiment, the present application includes a method of treating or preventing a viral infection comprising administering a therapeutically effective amount of one or more fractions of the application to a subject in need. In and embodiment, the one or more fractions of the application are administered in combination with another known agent useful for treating or preventing a viral infection.

In an embodiment, the known agent useful for treating or preventing a viral infection is selected from an antiviral, an antiretroviral, a corticosteroid, an antimalarial, an antibiotic and an immunotherapy, and a combination thereof. In an embodiment, the antiviral is selected from remdesivir, peramivir, zanamivir, oseltamivir and balozavir, and a combination thereof. In an embodiment, the antiretroviral is selected from lopinavir and ritonavir, and a combination thereof. In an embodiment, the corticosteroid is selected from cortisone, prednisone, and methylprednisolone, and a combination thereof. In an embodiment, the antimalarial is chloroquine. In an embodiment, the antibiotic is selected from a macrolide antibiotic, a fluoroquinolone, and a tetracycline antibiotic and a combination thereof. In an embodiment, the macrolide antibiotic is selected from azithromycin and clarithromycin, and a combination thereof. In an embodiment, the fluoroquinolone antibiotic is selected from ciprofloxaxin and levofloxacin, and a combination thereof. In an embodiment, the tetracycline is selected from doxycycline and tetracycline, and a combination thereof.

In an embodiment, the immunotherapy is an antibody therapy or a vaccine. In an embodiment, the antibody therapy is casirivimab, imdevimab or bamlanivimab, or combinations thereof. In an embodiment, the antibody therapy is casirivimab or imdevimab, or combinations thereof. In an embodiment, the antibody therapy is casirivimab and imdevimab. In an embodiment, the antibody therapy is bamlanivimab.

In an embodiment, the immunotherapy is a vaccine. In an embodiment, the vaccine is Covid-19 vaccine. In an embodiment, the Covid-19 vaccine is any Covid-19 approved or in development. In an embodiment, the vaccine is BNT162b2, AZD1222, Ad5-nCoV, mRNA-1273, CoronaVac, Gam-Covid-Vac, Ad26.COV2.S, NVX-CoV237, BBV152, CoVLP, CVnCoV, INO-4800, EpiVacCorona, INO-4800, AG0301-COVID-19, LV-SMENP-DC, LNP-nCoVsaRNA, GX-19, SCB-2019, COVAX-19, Lunar-COV19/ARCT-021, CoVLPs, COVID-19/aAPC, SARS-CoV-2 Sclamp, GRAd-Cov2, AD5-nCOV, Sputnik V or combinations thereof. In an embodiment, the vaccine is Ad26.COV2.S. In an embodiment, the vaccine is BNT162b2. In an embodiment, the vaccine is AZD1222. In an embodiment, the vaccine is mRNA-1273.

The one or more one or more fractions of the application can improve the efficacy of a vaccine for treating a viral infection. Accordingly, the one or more fractions of the application are useful as vaccine adjuvants.

Accordingly, the present application includes a method of improving the efficacy of a vaccine for treating a viral infection comprising administering a therapeutically effective amount of one or more fractions of the application to a subject in need thereof.

The present application also includes a use of one or more fractions of the application for improving the efficacy of a vaccine for treating a viral infection as well as a use of one or more fractions of the application for the preparation of a medicament for improving the efficacy of a vaccine for treating a viral infection. The application further includes one or more fractions of the application for improving the efficacy of a vaccine for treating a viral infection.

In an embodiment, one or more fractions of the application are administered with another agent simultaneously or sequentially in separate unit dosage forms or together in a single unit dosage form.

In an embodiment, the one or more fractions of the application is administered or used as soon as possible after exposure or possible exposure to the virus. In an embodiment, one or more fractions of the application is administered or used until treatment of the viral infection is achieved. For example, until complete elimination of the virus is achieved, or until the number of viruses has been reduced to the point where the subject's defenses are no longer overwhelmed and can kill any remaining viruses.

Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom.

In an embodiment, the one or more fractions of the application may be administered at least once a week. In an embodiment, the one or more fractions may be administered to the subject from about one time per three weeks, or about one time per week to about once daily for a given treatment. In another embodiment, the one or more fractions are administered 2, 3, 4, 5 or 6 times daily. The length of the treatment period depends on a variety of factors, such as the severity of the disease, disorder or condition, the age of the subject, the concentration and/or the activity of the one or more fractions of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration may be required. For example, the one or more fractions are administered to the subject in an amount and for duration sufficient to treat the patient.

Fractions of the application may be used alone or in combination with other known agents useful for treating diseases, disorders or conditions treatable by activation of TLR function, by stimulation of the production of one or more chemokines and/or associated with activating an innate and/or adaptive innate response such as a viral infection. When used in combination with other agents useful in treating diseases, disorders or conditions by activation of TLR function, by stimulation of the production of one or more chemokines and/or associated with activating an innate and/or adaptive innate response, it is an embodiment that the one or more fractions of the application are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, one or more fractions of the present application is administered with another therapeutic agent simultaneously or sequentially in separate unit dosage forms or together in a single unit dosage form. Accordingly, the present application provides a single unit dosage form comprising one or more fractions of the application, an additional therapeutic agent, and a pharmaceutically acceptable carrier.

The dosage of the one or more fractions of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Fractions of the application may be administered initially in a suitable dosage that may be adjusted as required, depending on the clinical response. Dosages will generally be selected to maintain a serum level of fractions of the application from about 0.01 μg/cc to about 1000 μg/cc, or about 0.1 μg/cc to about 100 μg/cc. As a representative example, oral dosages of one or more fractions of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the one or more fractions are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Fractions of the application may be administered in a single daily, weekly or monthly dose or the total daily dose may be divided into two, three or four daily doses.

In an embodiment, the fractions of the application and/or compositions thereof are either used or administered alone in the methods or uses described herein or in combination with other known agents used in the methods or uses described herein, for example, for treating a viral infection.

In an embodiment, the fractions of the application and/or compositions thereof are useful as additives to, for example, a nutraceutical composition. Therefore, the present application also includes the use of one or more fractions of the application and/or compositions thereof as additives.

In an embodiment, the subject is a mammal. In an embodiment, the subject is human.

### V. EXAMPLES

The following non-limiting examples are illustrative of the present application.

### Example 1: Preparation and Analysis of PBG-005 and PBG-007

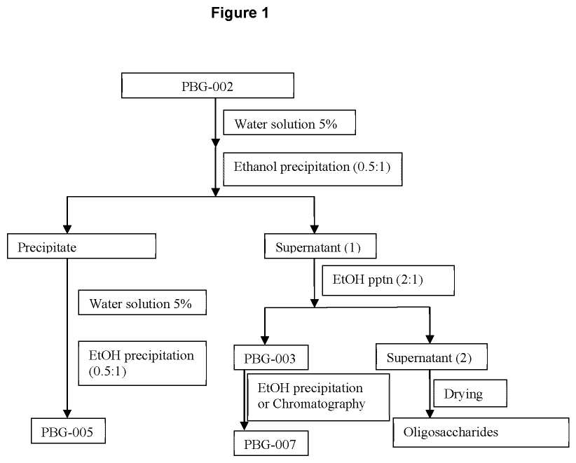

A flowchart showing an embodiment of a process of isolating intermediate polysaccharide fraction (PBG-002) and neutral polysaccharide fraction (PBG-005), acidic polysaccharide rich fraction and acidic polysaccharide fraction (PBG-007 therefrom is shown in FIG. 1.

### Step 1. Preparation of Intermediate Polysaccharide Fraction PBG-002

Milled North American ginseng root (20-40 mesh) was treated with 85% ethanol at the ratio of ethanol to ginseng raw material of 10:1 (V/w) under reflux for 3 hrs. The extract solution was separated, and the residue was extracted with water at the ratio of water to original ginseng raw material of 12:1 (V/W) under reflux for 3 hrs. The extract solution was separated by centrifugation, concentrated, and spray dried to give PBG-002 bulk powder.

### Step 2. Preparation of Neutral Polysaccharide Fraction PBG-005

To a solution of PBG-002 (5%) in water was added ethanol at the ratio of ethanol to solution of 0.5:1. The mixture was kept at room temperature for 3 hrs. The precipitate was separated by centrifugation. The precipitate was re-dissolved in water (5%) and undergone the same ethanol precipitation procedure to produce the neutral fraction PBG-005. The yield of PBG-005 from PBG-002 was around 10-15% with the purity of greater than 95%.

### Step 3. Preparation of Acidic Polysaccharide Rich Fraction PBG-003 and Acidic Polysaccharide Fraction PBG-007.

To the supernatant from first ethanol precipitation in neutral fraction preparation was added another portion of ethanol at the ratio of ethanol to supernatant of 2:1 (V/V). The mixture was kept at room temperature for 3 hrs. The precipitate was separated by centrifugation and dried to give acidic polysaccharide enriched fraction PBG-003. The acidic polysaccharide enriched fraction was then dissolved in water (5%) and ethanol was added to the solution at the ratio of 0.5:1. The mixture was stirred and kept at room temperature for 3 hrs. The precipitate was separated by centrifugation. The supernatant was concentrated and lyophilized to give dry powder acidic polysaccharide PBG-007-1. The yield of acidic polysaccharide fraction PBG-007 (PBG-007-1) from PBG-002 was around 10-15%, with the purity of greater than 85%.

### Step 4. Chromatographic Purification of Acidic Fraction to Produce Acidic Polysaccharide Fraction PBG-007

The acidic polysaccharide enriched fraction was then further purified by ion-exchange chromatography. The starting material of PBG-003 was dissolved in Milli-Q water at 50 mg/ml concentration first and then filtered through a 0.45 μm syringe filter unit before subject to HPLC. The solution was injected into Agilent 1200 prep HPLC system equipped with ELSD detector and FoxyR2 fraction collector in multiple injection method (5 injections per run and 5 ml injection volume per injection). Detail chromatographic conditions are as follows,

| Instrument: Agilent 1200 series prep HPLC system | |
|---|---|
| Column: TSK gel 13 μm DEAE-5PW column, 150 mm × | |
| 21.5 mm (54.5 ml) | |
| Detector: Agilent 1200 series ELSD (60° C., 3.5 bar) | |
| Eluent A: 20 mM NH₄OAc | Eluent B: 1M NH₄OAc |
| Flow rate: 3 ml/min | Injection volume: 5 mL |
| Runtime: 110 minutes | Post time: 2 min |

The desired fractions (retention time from 42 minutes to 100 min) were collected and combined for lyophilization to give dry powder as product (PBG-007-2) at the yield of around 10-15% from PBG-002 with the purity of greater than 95%.

### Example 2: Structural Characterization of Neutral and Acidic Polysaccharides

### 1. Component Sugar Analysis

Component sugar composition of the isolated neutral and acidic polysaccharides was determined by hydrolysis of polysaccharide chain to release its component sugars and further quantitation of the released component sugars. Table 1 lists the composition of typical samples from above process. Composition for crude polysaccharide is also listed as comparison.

TABLE 1
Component sugar composition of typical
acidic and neutral polysaccharide

| | Component Sugar (%) | | | | |
|---|---|---|---|---|---|
| Sample | Rha | GalA | Glc | Gal | Ara |
| PBG-002 | 1.27 | 15.13 | 72.43 | 5.27 | 5.62 |
| PBG-005 | 0.61 | 5.57 | 89.80 | 1.92 | 2.00 |
| PBG-007 | 4.04 | 45.56 | 12.18 | 19.81 | 18.20 |

### 2. Glycosyl Linkage Analysis

### Glycosyl Linkage (NaOH Method):

For the glycosyl linkage analysis, the sample was permethylated, depolymerized, reduced, and acetylated; and the resultant partially methylated alditol acetates (PMAAs) analyzed by gas chromatography-mass spectrometry (GC-MS) as described, for example, by York et al (1985) Methods Enzymol. 118:3-40.

Initially, an aliquot of the sample after dialysis was suspended in about 200 μl of dimethyl sulfoxide. The samples were then permethylated by the method of Ciukanu and Kerek (1984) Carbohydr. Res. 131:209-217 (treatment with sodium hydroxide and methyl iodide in dry DMSO). The sample was subjected to NaOH base for 10 minutes then methyl iodide was added and left for 20 minutes. The base was then added for 10 minutes, and finally more methyl iodide was added for 20 minutes. This addition of more methyl iodide and NaOH base was to insure complete methylation of the polymer. Following sample workup, the permethylated material was reduced by superdeuteride to reduce methyl ester of uronic acid, and then hydrolyzed using 2 M trifluoroacetic acid (2 h in sealed tube at 121° C.), reduced with NaBD4, and acetylated using acetic anhydride/trifluoroacetic acid. The resulting PMAAs were analyzed on a Hewlett Packard 5890 GC interfaced to a 5970 MSD (mass selective detector, electron impact ionization mode); separation was performed on a 30 m Supelco 2330 bonded phase fused silica capillary column.

### Glycosyl Linkage (Hakamori Method)

For glycosyl linkage analysis, the sample was methylated by a modification of the method of Hakomori; depolymerized, reduced, and acetylated; and the resultant partially methylated alditol acetates (PMAAs) analyzed by gas chromatography-mass spectrometry (GC-MS) as described, for example, by York et al (1985) Methods Enzymol. 118:3-40.

Briefly, an aliquot was taken from the sample after lyophilizing, suspended in about 200 μl of dimethyl sulfoxide and placed on a magnetic stirrer for 5 days. 0.7 mL potassium dimesylate (3.6 M) was added. After 8 hours at room temperature on stirrer, the reaction mixture was cooled to 0° C., excess methyl iodide (0.7 mL) was added, and the tube sealed. Incubation was then continued for overnight at room temperature. Following sample workup, the sample was dried under nitrogen, and added about 200 uL of super-deuteride incubating at room temperature for 1 hour and 45 mins, followed bypass through on-guard H cartridge. After drying under nitrogen, the sample was permethylated again by the method of Ciukanu and Kerek (1984) Carbohydr. Res. 131:209-217 (treatment with sodium hydroxide and methyl iodide in dry DMSO). The sample was subjected to the NaOH base for 10 minutes then methyl iodide was added and left for 40 minutes. More NaOH base and methyl iodide was then added for 40 minutes. This addition of more methyl iodide and NaOH base was to insure complete methylation of the polymer. The permethylated material was hydrolyzed using 2 M trifluoroacetic acid (2 h in sealed tube at 100° C.), reduced with NaBD4, and acetylated using acetic anhydride/pyridine. The resulting PMAAs were analyzed on a Hewlett Packard 5890 GC interfaced to a 5970 MSD (mass selective detector, electron impact ionization mode); separation was performed on a 30 m Supelco 2330 bonded phase fused silica capillary column.

### Results

Table 2 and Table 3 summarize the glycosyl linkage information of typical neutral and acidic polysaccharide samples obtained from above process.

TABLE 2
Glycosyl Linkage of Neutral Polysaccharide

| | PBG-005 |
|---|---|
| | Percentage |
| Glycosyl Residue | Present |
| Terminally linked arabinofuranosyl residue(t-Araf) | 0.5 |
| Terminally linked glucopyranosyl residue(t-Glcp) | 5.4 |
| 4-linked mannopyranosyl residue(4-Manp) | 0.8 |
| 4-linked glucopyranosyl residue(4-Glcp) | 91.6 |
| 4,6-linked glucopyranosyl residue(4,6-Glcp) | 1.6 |

TABLE 3
Glycosyl Linkage of Neutral Polysaccharide

| | PBG-007 |
|---|---|
| | Percentage |
| Glycosyl Residue | Present |
| Terminally linked rhaminopyranosyl residue (t-Rha) | 3.7 |
| Terminally linked arabinofuranosyl residue(t-Araf) | 4.5 |
| 2-linked rhaminopyranosyl residue (2-Rha) | 2.6 |
| Terminally linked glucopyranosyl residue(t-Glcp) | 4.9 |
| Terminally linked galactopyranosyl residue & terminally | 13.7 |
| linked galacturonic acid pyranosyl residue (t-Galp & t-Gal A) | |
| 4-linked arabinopyranosyl residue or 5-linked | 6.4 |
| arabinofuranosyl residue(4-Arap or 5-Araf) | |
| 2,4-linked rhaminopyranosyl residue (2,4-Rha) | 2.4 |
| 3-linked galactopyranosyl residue (3-Galp) | 2.5 |
| 4-linked galactopyranosyl residue & 4-linked galacturonic | 32.9 |
| acid pyranosyl residue(4-Galp & 4-Gal A) | |
| 4-linked glucopyranosyl residue(4-Glcp) | 24.0 |
| 2,4-linked galactopyranosyl residue (2,4-Galp) | 1.3 |
| 3,6-linked galactopyranosyl residue (3,6-Galp) | 1.2 |

As it is shown above, neutral polysaccharide PBG-005 contains mainly glucose as its structural component with over 90% of 4-glucose plus other minor linkages. Acidic polysaccharide PBG-007 contains galacturonic acid, arabinose, rhamnose, galactose and glucose as component sugars with galacturonic acid as major component. It contains mainly terminal GalA, 4-GalA, 4-Glc and other minor linkages.

### Example 3: Toll-like Receptor Activity

### Methods:

HEK293 cells are constructed to stably express specific human TLRs (TLR2 or TLR4). In this experiment, HEK293 cells were treated with polysaccharide fractions at concentration of 100, 500 or 2000 μg/mL for 24 hours. Output measurement for stimulation of IL-8 production by TLR2 and TLR4 in supernatant was measured by ELISA.

Positive Controls: Pam3CSK4 for TLR2 and LPS for TLR4

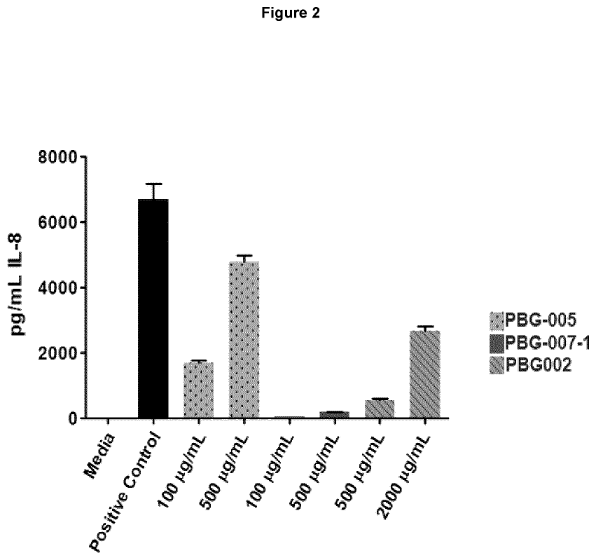

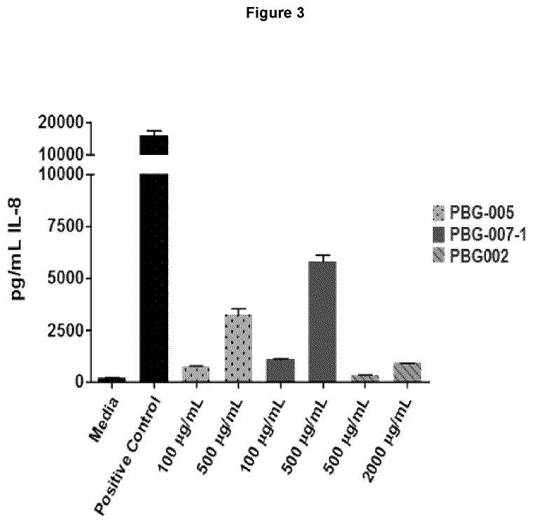

In this study, PBG-005 shows mixed activity affecting both TLR2 and TLR4 while PBG-007-1 displayed minimal TLR2 activity and strong TLR4 activity (see FIGS. 2 and 3). PBG-002 was more active on TLR2 than TLR4. Comparing the three exemplary fractions of the application with respect to TLR2 activity: PBG-005>PBG-002>PBG-007-1. With respect to TLR4 activity: PBG-007-1>PBG-005>PBG-002

### Example 4: Action on H1N1 Influenza Virus

### Macrophages (In Vitro)

### Methods:

Primary human macrophages were pre-treated with PBG-002, PBG-005, PBG-007-1 or media alone at varying concentrations for 24 hours. In presence of the exemplary fractions of the application, virus was added at MOI of 0.1 and cells were cultured for 40 minutes. Virus was then washed off and fresh media and exemplary fractions of the application were added and cultured for 24 h. Supernatant was collected and viral titre was determined by adding dilutions of the supernatant to a susceptible cell line.

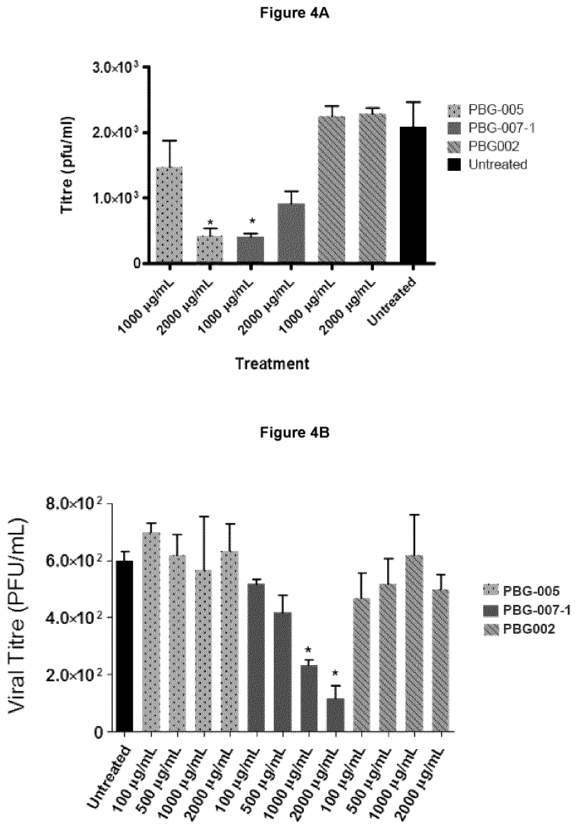

Discussion: The first study indicated that both PBG-005 and PBG-007-1 had a significant impact on H1N1 titre, albeit with an unclear dose response profile, while PBG-002 had no impact on the titre (See FIGS. 4A and 4B). A follow up study with a higher number of doses demonstrated no activity of with PBG-005 or PBG-002 on viral titre, which contradicted the PBG-005 result from the earlier replication. PBG-007-1 displayed a clear dose response impact on H1N1 titre, which reached significance only at the two highest doses.

### Example 5: Action on HSV-1

### a) Mouse Dermal Cells (In Vitro)

### Methods:

Primary mouse dermal fibroblasts were pre-treated with PBG-002, PBG-005, PBG-007-1 or media alone at varying concentrations for 24 hours. In presence of the exemplary fractions of the application, virus was added at MOI of 0.1 and cells were cultured for 40 minutes. Virus was then washed off and fresh media and the exemplary fractions of the application were added and cultured for 24 h. Supernatant was collected, and viral titre was determined by adding dilutions of the supernatant to a susceptible cell line

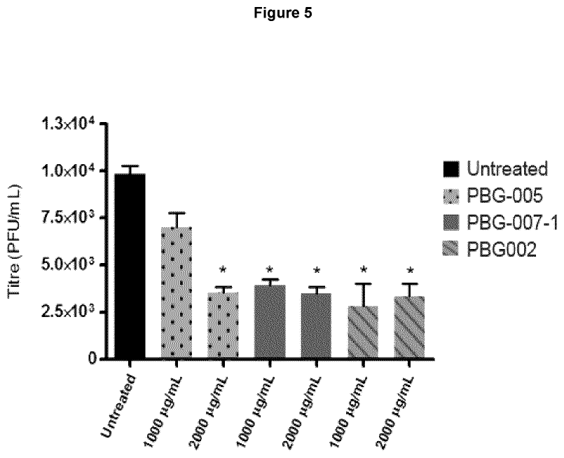

Discussion: In mouse dermal fibroblasts, all three exemplary fractions of the application significantly decreased the HSV-1 titre at 2000 μg/mL and both PBG-007-1 and PBG-002 displayed similar effects at 1000 μg/mL (see FIG. 5).

### b) Human Skin Fibroblasts (In Vitro)

### Methods:

Human skin fibroblast cell line cells were pre-treated with PBG-002, PBG-005, PBG-007-1 or media alone at varying concentrations for 24 hours. In presence of the exemplary fractions of the application, virus was added at MOI of 0.1 and cells were cultured for 40 minutes. Virus was then washed off and fresh media and exemplary fractions of the application were added and cultured for 24 h. Supernatant was collected, and viral titre was determined by adding dilutions of the supernatant to a susceptible cell line

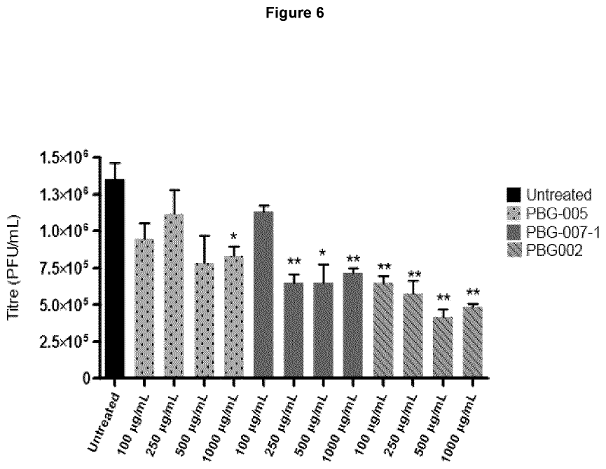

Discussion: In human skin fibroblast cells, PBG-005 had a significant impact on HSV-1 titre at only the highest concentration while PBG-007-1 had a significant impact at all but the lowest dose and PBG-002 significantly decreased HSV-1 titre at all concentrations (see FIG. 6).

### Example 6: Action on HSV-2

### a) Mouse Dermal Cells (In Vitro)

### Methods

Primary mouse dermal fibroblasts were pre-treated with PBG-002, PBG-005, PBG-007-1 or media alone at varying concentrations for 24 hours. In presence of the exemplary fractions of the application, virus was added at MOI of 0.1 and cells were cultured for 40 minutes. Virus was then washed off and fresh media and exemplary fractions of the application were added and cultured for 24 h. Supernatant was collected, and viral titre was determined by adding dilutions of the supernatant to a susceptible cell line

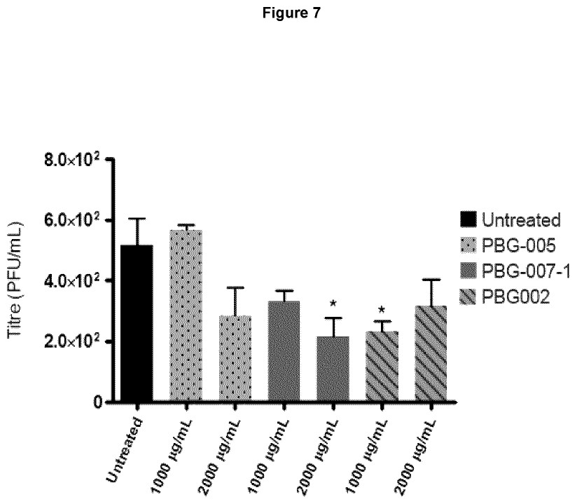

Discussion: In the context of HSV-2 infection of mouse dermal fibroblasts, both PBG-007-1 and PBG-002 had a significant impact on HSV-2 titre (see FIG. 7).

### b) Human Skin Fibroblasts (In Vitro)

### Methods:

Human skin fibroblast cell line cells were pre-treated with PBG-002, PBG-005, PBG-007-1 or media alone at varying concentrations for 24 hours. In presence of the exemplary fractions of the application, virus was added at MOI of 0.1 and cells cultured for 40 min. Virus was washed off and fresh media and exemplary fractions of the application were added and cultured for 24 h. Supernatant was collected, and viral titre was determined by adding dilutions of the supernatant to a susceptible cell line

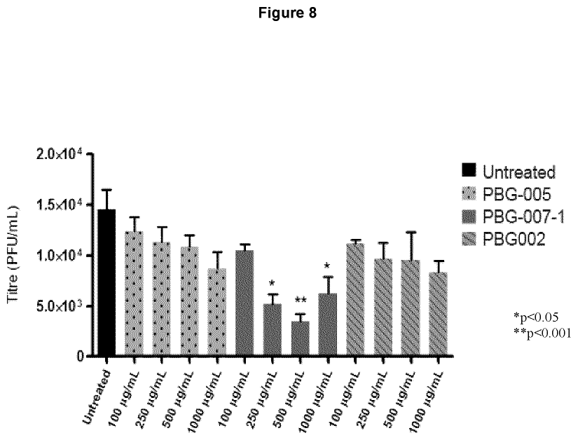

Discussion: In a human skin fibroblast cell line infected with HSV-2, PBG-002 had no significant impact on HSV-2 titre. PBG-005 had an apparent dose dependent impact on HSV-2 titre but it did not reach statistical significance. PBG-007-1 significantly decreased viral titre at the three highest doses but there was no clear dose dependency of the effect (see FIG. 8).

### c) Human Foreskin Fibroblasts (In Vitro)

### Methods:

Primary human foreskin fibroblasts were pre-treated with PBG-002, PBG-005, PBG-007-1 or media alone at varying concentrations for 24 hours. In presence of the exemplary fractions of the application, virus was added at MOI of 0.1 and cells were cultured for 40 min. Virus was then washed off and fresh media and exemplary fractions of the application were added and cultured for 24 h. Supernatant was collected, and viral titre was determined by adding dilutions of the supernatant to a susceptible cell line.

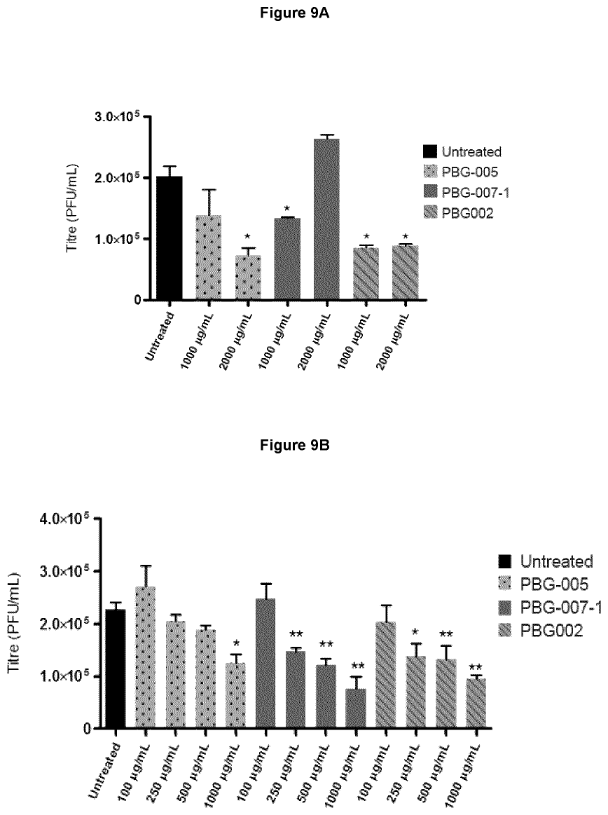

Discussion: In the first study looking at HSV-2 titre in human foreskin fibroblast cultures, all three exemplary fractions of the application significantly decreased the HSV-2 titre but with different patterns. PBG-005 had a significant impact at 2000 μg/mL while PBG-002 significantly decreased viral titre at both concentrations. PBG-007-1 significantly decreased HSV-2 titre at 1000 μg/mL but not at 2000 μg/mL. In the second study, both PBG-002 and PBG-007-1 caused a dose dependent decrease in viral titre that was significant at 250 μg/mL and above. PBG-005 only impacted the HSV-2 titre at 1000 μg/mL (see FIGS. 9A and 9B).

### d) Guinea Pig Model (In Vivo)

### Methods:

Female Hartley strain guinea pigs were randomized into 4 groups (N=5). Exemplary fractions of the application were resuspended to a 5 mg/ml stock in sterile water and incubated at 37 C for 1 hour. Animals were given 10 mg/d of the compound, administered orally drop-wise onto the back of the tongue. Animals were treated for 7 days prior to HSV-2 infection, on the day of infection, for 12 days after infection (primary infection stage) and every other day after recovery from primary infection. Animals were inoculated intravaginally with 10⁵ PFU HSV-2. Animals were evaluated daily for genital lesions, weight loss, urinary retention, hindlimb paralysis, and body condition. Virus-induced lesions on the external genital skin were scored using this scale: 0, no apparent inflammation and infection; 1+, slight redness of external vaginal orifice or swelling; 2+, one to four small vesicles; 3+, five to nine large vesicles; 4+, greater than nine large ulcers with maceration; and 5, hindlimb paralysis. In addition, guinea pigs were scored 0.5, 1.5, 2.5, or 3.5 when intermediate in lesion severity.

Animals were euthanized prior to the completion of the study if there was a body weight loss of greater than 25 percent, when a lesion score of 4 persisted for more than nine days, full hindlimb paralysis occurred or if hindlimb weakness did not reverse within two days. All animals were euthanized eighty days post HSV-2 challenge.

Vaginal secretions were collected from guinea pigs for 10 days following HSV-2 IVAG challenge. Specimens were stored frozen and viral titres later determined by a plaque assay.

### DISCUSSION

Viral titre during the primary infection was lower in both the PBG-005 and PBG-007-1 groups than the control and PBG-002 groups. The vaginal lesion scores during the primary infection period were highest in the control group and lowest in the PBG-007-1 group.

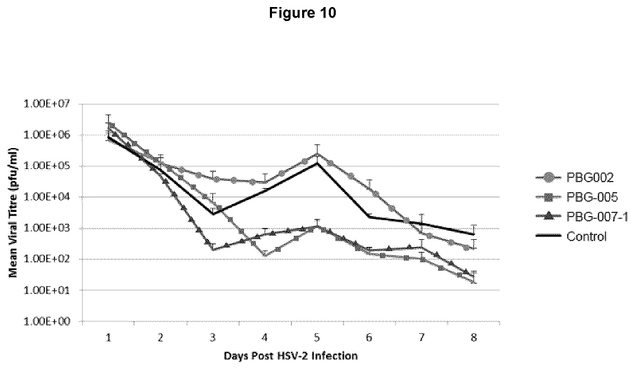

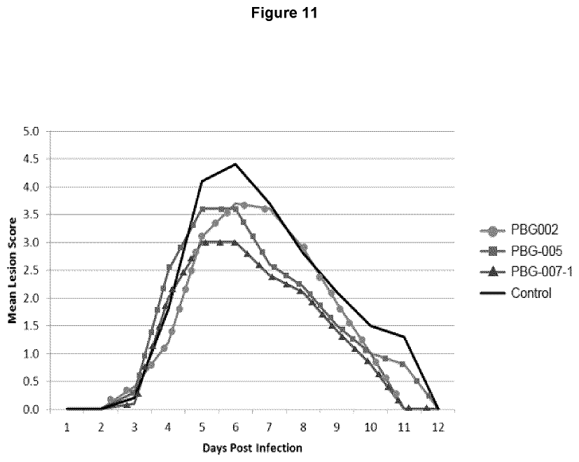

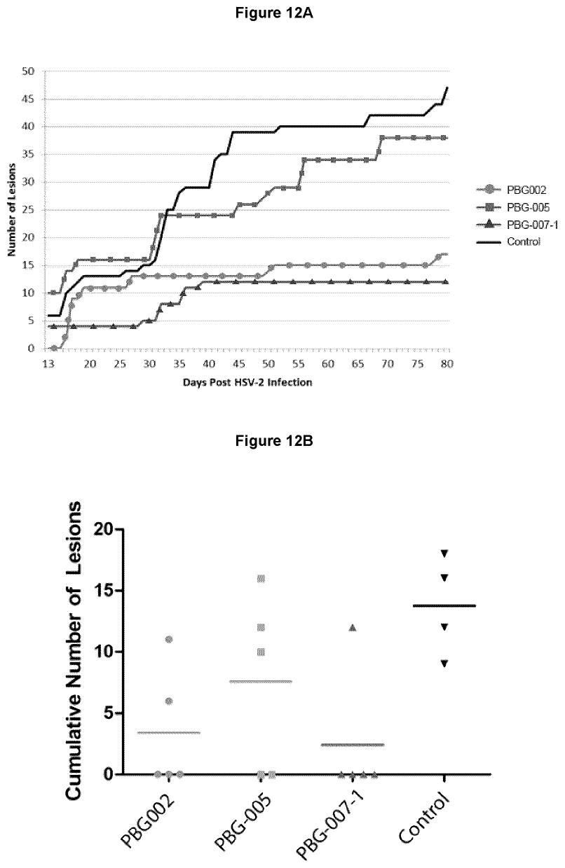

The total cumulative lesion scores after the primary infection period were lowest in the PBG-002 and the PBG-007-1 groups and highest in the control group. When cumulative lesion number was plotted by individual animal, all animals in the control group (4/4; one animal in this group did not survive the primary infection) had vaginal lesions compared with 3/5 animals in the PBG-005 group, 2/5 animals in the PBG-002 group and 1/5 animal in the PBG-007-1 group (See FIGS. 10, 11 and 12A and 12B)

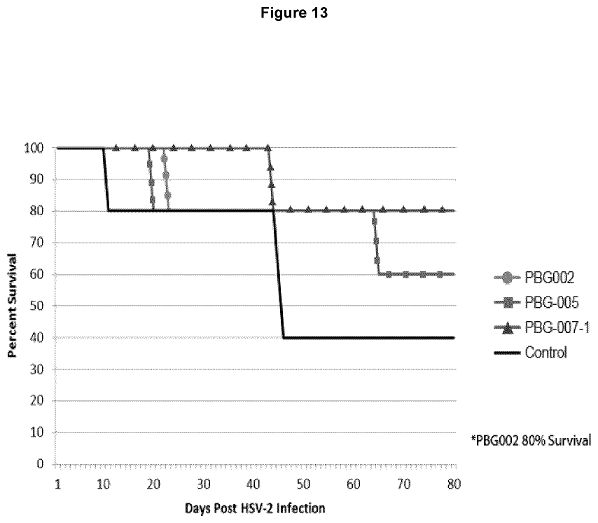

The survival data is inconclusive since the groups only had 5 mice each, so each death is a drop of 20%. PBG-002 and PBG-007-1 had the same survival rate (80%), but the PBG-007-1 maintained 100% survival for longer. The control group was the first group to lose an animal and had the fewest animals surviving at the end of the study (40%). PBG-005 had a moderate rate of survival (60%) (see FIG. 13).

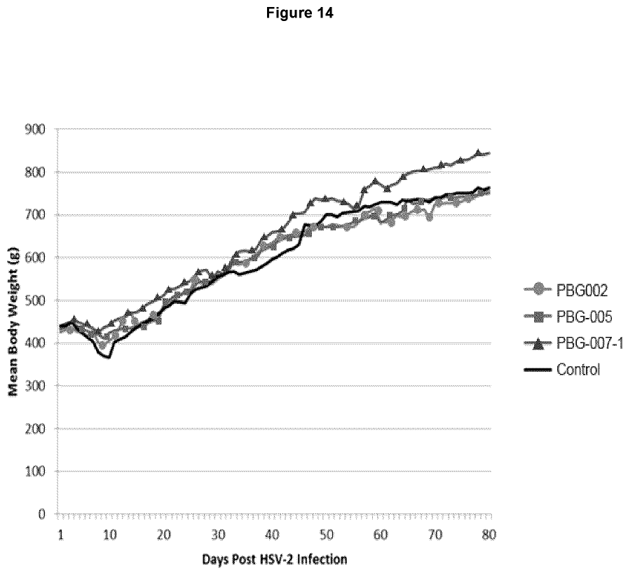

Loss of body weight is an indication of serious illness in these animals, so their body weight was recorded to determine whether euthanasia was required. The PBG-007-1 group had the highest mean body weight (see FIG. 14).

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

A number of publications are cited herein. Full citations for these references are provided below. Each of these references is incorporated herein by reference in its entirety into the present disclosure, to the same extent as if each individual reference was specifically and individually indicated to be incorporated by reference
Akira S, Uematsu S, Takeuchi O., Pathogen recognition and innate immunity, ell. 2006 Feb. 24; 124(4):783-801.
Hargreaves D C1, Medzhitov R., Innate sensors of microbial infection, J Clin Immunol. 2005 November; 25(6):503-10.
Kawai T, Akira S, Antiviral signaling through pattern recognition receptors, J Biochem. 2007 February; 141(2):137-45. Epub 2006
Philpott D J1, Girardin S E., The role of Toll-like receptors and Nod proteins in bacterial infection, Mol Immunol. 2004 November; 41(11):1099-108.
Seth R B, Sun L, Chen Z J., Antiviral innate immunity pathways, Cell Res. 2006 February; 16(2):141-7.
Janeway C A Jr, Medzhitov R., Introduction: the role of innate immunity in the adaptive immune response, Semin Immunol. 1998 October; 10(5):349-50.
Janeway C A Jr, The immune system evolved to discriminate infectious nonself from noninfectious self, Immunol Today. 1992 January; 13(1):11-6.
Janeway C A Jr, Medzhitov R., Lipoproteins take their toll on the host, Curr Biol. 1999 Dec. 2; 9 (23):R879-82
Akira S, Takeda K., Toll-like receptor signalling, Nat Rev Immunol. 2004 July; 4 (7):499-511.
O'Neill L A, How Toll-like receptors signal: what we know and what we don't know, Curr Opin Immunol. 2006 February; 18(1):3-9.
Iwasaki A1, Medzhitov R., Toll-like receptor control of the adaptive immune responses, Nat Immunol. 2004 October; 5(10):987-95.
Bauer S1, Kirschning C J, Häcker H, Redecke V, Hausmann S, Akira S, Wagner H, Lipford G B., Human TLR9 confers responsiveness to bacterial DNA via species-specific CpG motif recognition, Proc Natl Acad Sci USA. 2001 Jul. 31; 98(16):9237-42.
Krug A1, Towarowski A, Britsch S, Rothenfusser S, Hornung V, Bals R, Giese T, Engelmann H, Endres S, Krieg A M, Hartmann G., Toll-like receptor expression reveals CpG DNA as a unique microbial stimulus for plasmacytoid dendritic cells which synergizes with CD40 ligand to induce high amounts of IL-12, EurJ Immunol. 2001 October; 31(10):3026-37.
Ashkar A A1, Rosenthal K L., Toll-like receptor 9, CpG DNA and innate immunity, Curr Mol Med. 2002 September; 2(6):545-56.
Nguyen N H1, Nguyen C T, Pharmacological effects of ginseng on infectious diseases, Inflammopharmacology. 2019 October; 27(5):871-883.

1. A process of preparing an acidic polysaccharide fraction from a root portion of an American ginseng (Panax quinquefolius) plant, the process comprising:
extracting the root portion of an American ginseng (Panax quinquefolius) plant with a solvent mixture comprising about 50% (v/v) to about 95% (v/v) C₁₋₃ alkylOH and water at a temperature of about 10° C. to about 90° C. to produce a solvent mixture fraction and a first residue, wherein the ratio of the solvent mixture to the root portion is about 20 (v/w) to about 1 (v/w) to about 1 (v/w) to about 1 (v/w),
separating the first residue from the solvent mixture fraction;
extracting the first residue with water at a temperature of about 10° C. to about 100° C. to produce an aqueous fraction comprising an intermediate polysaccharide fraction and a second residue, wherein the ratio of the water to the root portion is about 20 (v/w) to about 1 (v/w) to about 1 (v/w) to about 1 (v/w),
separating the aqueous fraction comprising the intermediate polysaccharide fraction from the second residue;
drying or concentrating the aqueous fraction to produce the intermediate polysaccharide fraction;
dissolving the intermediate polysaccharide fraction in water to produce an intermediate polysaccharide fraction solution wherein the intermediate polysaccharide fraction solution is about 1% (w/w) to about 10% (w/w),
adding C₁₋₃ alkylOH to the intermediate polysaccharide fraction solution to produce a neutral polysaccharide fraction and a first supernatant wherein the ratio of the C₁₋₃ alkylOH to the intermediate polysaccharide fraction solution is about 1(v/v) to about 1 (v/v) to about 0.5 (v/v) to about 1 (v/v);
separating the neutral polysaccharide fraction from the first supernatant to produce the neutral polysaccharide fraction;
adding C₁₋₃ alkylOH to the first supernatant to produce an acidic polysaccharide rich fraction and a second supernatant wherein the ratio of the C₁₋₃ alkylOH to first supernatant is about 0.5 (v/v) to about 1 (v/v) to about 8 (v/v) to about 1 (v/v),
separating the acidic polysaccharide rich fraction from the second supernatant,
dissolving the acidic polysaccharide rich fraction in water to produce an acidic polysaccharide rich fraction solution wherein the acidic polysaccharide rich fraction solution is about 1% (w/w) to about 10% (WM);
adding C₁₋₃ alkylOH to the acidic polysaccharide rich fraction solution to produce a precipitate and a third supernatant comprising an acidic polysaccharide fraction wherein the ratio of C₁₋₃ alkylOH to the acidic polysaccharide rich fraction solution about 1 (v/v) to about 1 (v/v) to about 0.5 (v/v) to about 1 (v/v);
separating the third supernatant comprising the acidic polysaccharide fraction from the precipitate; and
optionally, drying or concentrating the third supernatant to produce the acidic polysaccharide fraction; or
loading the acidic polysaccharide rich fraction solution on a chromatographic column comprising the anion exchange resin;
eluting the acidic polysaccharide rich eluent fraction from the anion exchange resin using an ammonium acetate buffer to produce the acidic polysaccharide eluent fraction, wherein the eluting comprises a first step wherein the buffer is about 10 mM to about 100 mM of ammonium acetate, and a second step wherein the buffer comprises about 0.5M to about 1.5M of ammonium acetate, and
optionally, drying or concentrating the acidic polysaccharide eluent fraction to produce the acidic polysaccharide fraction.

2. (canceled)

3. (canceled)

4. (canceled)

5. The process of claim 1, wherein the solvent mixture in the step of extracting the root portion comprises about 80% C₁₋₃ alkylOH (v/v) to about 90% C₁₋₃ alkylOH (v/v).

6. (canceled)

7. (canceled)

8. The process of claim 1, wherein the ratio of the solvent mixture to the root portion is about 10 (v/w) to about 1 (v/w).

9. (canceled)

10. (canceled)

11. (canceled)

12. The process of claim 1, wherein the ratio of the water to the root portion in the step of extracting the first residue is about 12 (v/w) to about 1 (v/w).

13. The process of claim 1, wherein the step of extracting the root portion of an American ginseng (Panax quinquefolius) plant with the solvent mixture or the step of extracting the first residue with water is performed at a temperature of about 20° C. to about 100° C., about 30° C. to about 100° C., about 40° C. to about 100° C., about 50° C. to about 100° C., about 60° C. to about 100° C., about 70° C. to about 100° C., about 80° C. to about 100° C. or about 90° C. to about 100° C. and performed over more than about 1 hour, more than about 2 hours, or more than about 3 hours.

14. (canceled)

15. (canceled)

16. The process of claim 1, wherein the ratio of C₁₋₃ alkylOH to the intermediate polysaccharide fraction solution in the step of adding C₁₋₃ alkylOH to the intermediate polysaccharide fraction solution is about 1 (v/v) to about 1 (v/v) to about 0.5 (v/v) to about 1 (v/v).

17. (canceled)

18. (canceled)

19. (canceled)

20. The process of claim 1, wherein C₁₋₃ alkylOH is added to the first supernatant in a ratio of 0.5 (v/v) to about 1 (v/v) to about 6 (v/v) to about 1 (v/v) of C₁₋₃ alkylOH to the first supernatant.

21. (canceled)

22. (canceled)

23. (canceled)

24. The process of claim 1, wherein the acidic polysaccharide rich fraction solution or the intermediate polysaccharide fraction solution is about 5% (w/w).

25. The process of claim 1, wherein C₁₋₃ alkylOH is added the acidic polysaccharide rich fraction solution in a ratio of about 0.7 (v/v) to about 1 (v/v) to about 0.5 (v/v) to about 1 (v/v) of C₁₋₃ alkylOH to the acidic polysaccharide rich fraction solution.

26. (canceled)

27. The process of claim 1, wherein each C₁₋₃ alkylOH is ethanol.

28. (canceled)

29. (canceled)

30. The process of claim 1, wherein the buffer in the first step of the eluting is about 20 mM ammonium acetate, and the buffer in the second step of the eluting is about 1M of ammonium acetate.

31. (canceled)

32. (canceled)

33. (canceled)

34. (canceled)

35. (canceled)

36. An acidic polysaccharide fraction produced by the process of claim 1.

37. An acidic polysaccharide fraction having a carbohydrate content comprising about 1 mole % to about 8 mole % rhamnose (Rha), about 40 mole % to about 50 mole % galacturonic acid (GalA), about 8 mole % to about 16 mole % glucose (Glc), about 14 mole % to about 25 mole % galactose (Gal) and about 15 mole % to about 25 mole % arabinose (Ara).

38. The acidic polysaccharide fraction of claim 37 which comprises about 2 wt % to about 5 wt % terminally linked rhaminopyranosyl residue (t-Rha), about 3 to wt % about 6 wt % terminally linked arabinofuranosyl residue (t-Araf), about 1 wt % to about 4 wt % 2-linked rhaminopyranosyl residue (2-Rha), about 3 wt % to about 6 wt % terminally linked glucopyranosyl residue (t-Glcp), about 12 wt % to about 15 wt % terminally linked galactopyranosyl residue and terminally linked galacturonic acid pyranosyl residue (t-Galp and t-Gal A), about 4 wt % to about 8 wt % 4-linked arabinopyranosyl residue or 5-linked arabinofuranosyl residue (4-Arap or 5-Araf), about 1 wt % to about 4 wt % 2,4-linked rhaminopyranosyl residue (2,4-Rha), about 1 wt % to about 4 wt % 3-linked galactopyranosyl residue (3-Galp), about 31 wt % to about 35 wt % 4-linked galactopyranosyl residue and 4-linked galacturonic acid pyranosyl residue (4-Galp and 4-Gal A), about 22 wt % to about 26 wt % 4-linked glucopyranosyl residue (4-Glcp), about 0.5 wt % to about 3 wt % 2,4-linked galactopyranosyl residue (2,4-Galp), and about 0.5 wt % to about 3 wt % 3,6-linked galactopyranosyl residue (3,6-Galp).

39. A composition comprising the acidic polysaccharide fraction of claim 37 and a carrier.

40. The composition of claim 39, wherein the composition is a pharmaceutical comprising a pharmaceutically acceptable carrier.

41. The composition of claim 39, wherein the composition is nutraceutical composition.

42. (canceled)

43. (canceled)

44. A method for activating an innate and/or subsequent adaptive response or for treating a disease, disorder or condition associated with activating an innate and/or adaptive innate response comprising administering a therapeutically effective amount of the acidic polysaccharide fraction of claim 37 to a subject in need thereof.

45. (canceled)

46. (canceled)

47. (canceled)

48. The method of 44, wherein the disease, disorder or condition is a viral infection, a microbial infection or a cancer.

49. The method of claim 48, wherein the disease, disorder or condition is a viral infection.

50. (canceled)

51. (canceled)
